SEQUENCE LISTING (77 sequences)

SEQ ID NO: 1 - protein, 2043 aa, synthetic construct
  REGION 1..2043: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKVSGGSPKKKRKVSGGSPKKKRKV

SEQ ID NO: 2 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 3 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 4 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAAAIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 5 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 6 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAAAIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 7 - protein, 1720 aa, synthetic construct
  REGION 1..1720: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 8 - protein, 1720 aa, synthetic construct
  REGION 1..1720: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 9 - protein, 1720 aa, synthetic construct
  REGION 1..1720: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 10 - protein, 1711 aa, synthetic construct
  REGION 1..1711: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 11 - protein, 1720 aa, synthetic construct
  REGION 1..1720: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINSGGSSGGSSGSETPGTSESATPESSGGSSGGSGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 12 - protein, 1716 aa, synthetic construct
  REGION 1..1716: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINGGGGSGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 13 - protein, 1716 aa, synthetic construct
  REGION 1..1716: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDYLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINGGGGSGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 14 - protein, 1716 aa, synthetic construct
  REGION 1..1716: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIFDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINGGGGSGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 15 - protein, 1805 aa, synthetic construct
  REGION 1..1805: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINGGGGSGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 16 - protein, 1954 aa, synthetic construct
  REGION 1..1954: note = Diversifying Base Editor
MKRTADGSEFEPKKKRKVSGGSSGGSANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESESGGSGGSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINGGGGSGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 17 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDRETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 18 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 19 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAAAIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDRETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 20 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAAAIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 21 - protein, 1904 aa, synthetic construct
  REGION 1..1904: note = Diversifying Base Editor
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDRETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSPKKKRKVSGGSPKKKRKVSGGSPKKKRKV

SEQ ID NO: 22 - protein, 1857 aa, synthetic construct
  REGION 1..1857: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINGGGGSGGGGSGGGGSGGGGSGGGGSMAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQSGGSSGGSSGSETPGTSESATPESSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDRETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 23 - protein, 1711 aa, synthetic construct
  REGION 1..1711: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 24 - protein, 1711 aa, synthetic construct
  REGION 1..1711: note = diversifying base editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDYLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 25 - protein, 1711 aa, synthetic construct
  REGION 1..1711: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIFDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 26 - protein, 1800 aa, synthetic construct
  REGION 1..1800: note = Diversifying Base Editor
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 27 - protein, 1954 aa, synthetic construct; identical to SEQ ID NO: 16
  REGION 1..1954: note = Diversifying Base Editor

SEQ ID NO: 28 - DNA, 1997 bp, Zea mays
  source 1..1997: mol_type = genomic DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 29 - DNA, 272 bp, Agrobacterium tumefaciens
  source 1..272: mol_type = other DNA
ccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtactcgacggccgagt

SEQ ID NO: 30 - DNA, 225 bp, Cauliflower mosaic virus
  source 1..225: mol_type = genomic DNA
tggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 31 - DNA, 64 bp, synthetic construct
  misc_feature 1..64: note = truncated glycine-tRNA
  source 1..64: mol_type = other DNA
ccagtggtctagtggtagaatagtaccctgccacggtacagacccgggttcgattcccggctgg

SEQ ID NO: 32 - DNA, 21 bp, synthetic construct
  misc_feature 1..21: note = 21-bp direct repeat sequence
  source 1..21: mol_type = other DNA
taatttctactaagtgtagat

SEQ ID NO: 33 - DNA, 24 bp, synthetic construct
  misc_feature 1..24: note = Protospacer for OsAAT
  source 1..24: mol_type = other DNA
ggttcgctcgacctgatcggtgct

SEQ ID NO: 34 - skipped

SEQ ID NO: 35 - DNA, 516 bp, Oryza sativa
  source 1..516: mol_type = genomic DNA
tttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagctt

SEQ ID NO: 36 - DNA, 18 bp, synthetic construct
  misc_feature 1..18: note = OsAAT PCR Primer
  source 1..18: mol_type = other DNA
atcctcggggtaagatcc

SEQ ID NO: 37 - DNA, 20 bp, synthetic construct
  misc_feature 1..20: note = OsAAT PCR Primer
  source 1..20: mol_type = other DNA
gaccacattcagcaccaggg

SEQ ID NO: 38 - RNA, 118 nt, synthetic construct
  misc_feature 1..118: note = MS2 crRNA1
  source 1..118: mol_type = other RNA
aattaacatgaggatcacccatgtctgcagggcctagaacttggccaacatgaggatcacccatgtctgcagttaatttctactaagtgtagatggttcgctcgacctgatcggtgct

SEQ ID NO: 39 - RNA, 103 nt, synthetic construct
  misc_feature 1..103: note = MS2 crRNA2
  source 1..103: mol_type = other RNA
aattaaacatgaggatcacccatgtctgcagaacatgaggatcacccatgtctgcagttaatttctactaagtgtagatggttcgctcgacctgatcggtgct

SEQ ID NO: 40 - RNA, 138 nt, synthetic construct
  misc_feature 1..138: note = MS2 crRNA3
  source 1..138: mol_type = other RNA
ttagagctaggccaacatgaggatcacccatgtctgcagggcctagcaagttaaaataaggccaacatgaggatcacccatgtctgcagggcctaatttctactaagtgtagatggttcgctcgacctgatcggtgct

SEQ ID NO: 41 - RNA, 142 nt, synthetic construct
  misc_feature 1..142: note = MS2 crRNA4
  source 1..142: mol_type = other RNA
taggcaacatgaggatcacccatgtctgcaggcctagcaagttaataaggcttcaacttggccaacatgaggatcacccatgtctgcagggccaagttaatttctactaagtgtagatggttcgctcgacctgatcggtgct

SEQ ID NO: 42 - protein, 342 aa, synthetic construct
  REGION 1..342: note = TadA9 with MS2-binding protein
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINKRPAATKKAGQAKKKK

SEQ ID NO: 43 - protein, 374 aa, synthetic construct
  REGION 1..374: note = hA3A with MS2-binding protein
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDYLYKEALQMLRDAGAQVSIMTYDEFKHCWDTFVDHQGCPFQPWDGLDEHSQALSGRLRAILQNQGNKRPAATKKAGQAKKKK

SEQ ID NO: 44 - protein, 351 aa, synthetic construct
  REGION 1..351: note = AID with MS2-binding protein
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSDPATFTYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLAEAGVQIAIMTFKDYFYCWNTFVENHERTFKAWEGLHENSVRLSRQLRRILQKRPAATKKAGQAKKKK

SEQ ID NO: 45 - protein, 10 aa, synthetic construct
  REGION 1..10: note = Linker
SGGSGGSGGS

SEQ ID NO: 46 - protein, 16 aa, synthetic construct
  REGION 1..16: note = Linker
SGSETPGTSESATPES

SEQ ID NO: 47 - protein, 32 aa, synthetic construct
  REGION 1..32: note = Linker
SGGSSGGSSGSETPGTSESATPESSGGSSGGS

SEQ ID NO: 48 - protein, 48 aa, synthetic construct
  REGION 1..48: note = Linker
SGSETPGTSESATPESSGGSSGGSSGSETPGTSESATPESSGGSSGGS

SEQ ID NO: 49 - protein, 17 aa, synthetic construct
  REGION 1..17: note = NLS
KRTADGSEFEPKKKRKV

SEQ ID NO: 50 - protein, 4 aa, synthetic construct
  REGION 1..4: note = Linker
SGGS

SEQ ID NO: 51 - protein, 5 aa, synthetic construct
  REGION 1..5: note = Linker
GGGGS

SEQ ID NO: 52 - protein, 1701 aa, synthetic construct
MKRTADGSEFEPKKKRKVSGGSSGGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 53 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtttccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 54 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacctgagccgaggtgaaattcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 55 - RNA, 65 nt, synthetic construct
  source 1..65: mol_type = other RNA
taatttctactaagtgtagatcaccctgacccacggcgtgcagttaatttctactaagtgtagat

SEQ ID NO: 56 - RNA, 65 nt, synthetic construct
  source 1..65: mol_type = other RNA
taatttctactaagtgtagatacctcggctcaggtcttgtagtttaatttctactaagtgtagat

SEQ ID NO: 57 - DNA, 1307 bp, Arabidopsis thaliana
  source 1..1307: mol_type = genomic DNA
gatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatcaggtgttagtttctagtttgtgcgatcgaatttgtcgattaatctgagtttttctgattaacag

SEQ ID NO: 58 - DNA, 446 bp, Arabidopsis thaliana
  source 1..446: mol_type = genomic DNA
ttcgttgaacaacggaaactcgacttgcctttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattg

SEQ ID NO: 59 - DNA, 528 bp, Cauliflower mosaic virus
  source 1..528: mol_type = genomic DNA
catggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 60 - RNA, 23 nt, synthetic construct
  source 1..23: mol_type = other RNA
tcctacctcatctgggacatcat

SEQ ID NO: 61 - RNA, 24 nt, synthetic construct
  source 1..24: mol_type = other RNA
aaagtgccaccacttgggatcatc

SEQ ID NO: 62 - RNA, 23 nt, synthetic construct
  source 1..23: mol_type = other RNA
gtcccttatttctcatggaaaat

SEQ ID NO: 63 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 64 - RNA, 24 nt, synthetic construct
  source 1..24: mol_type = other RNA
gttgatgttcctaaggatattcag

SEQ ID NO: 65 - RNA, 24 nt, synthetic construct
  source 1..24: mol_type = other RNA
gtgttaggtttgatgaccgtgtca

SEQ ID NO: 66 - RNA, 24 nt, synthetic construct
  source 1..24: mol_type = other RNA
ccgtgacacggtcatcaaacctaa

SEQ ID NO: 67 - RNA, 24 nt, synthetic construct
  source 1..24: mol_type = other RNA
tagaaccgatcttcccattgcatg

SEQ ID NO: 68 - RNA, 24 nt, synthetic construct
  source 1..24: mol_type = other RNA
aaagtgccaccacttgggatcatc

SEQ ID NO: 69 - RNA, 24 nt, synthetic construct
  source 1..24: mol_type = other RNA
gcgtctcttggaaggcgtcagtac

SEQ ID NO: 70 - RNA, 24 nt, synthetic construct
  source 1..24: mol_type = other RNA
attgcatgaccatcccaagatgct

SEQ ID NO: 71 - RNA, 24 nt, synthetic construct
  source 1..24: mol_type = other RNA
tgcagatgcttggcatgcacggga

SEQ ID NO: 72 - RNA, 24 nt, synthetic construct
  source 1..24: mol_type = other RNA
ggaggtgcctccatggagatccac

SEQ ID NO: 73 - RNA, 24 nt, synthetic construct
  source 1..24: mol_type = other RNA
taacgtcctcccccgtcacgaaca

SEQ ID NO: 74 - RNA, 24 nt, synthetic construct
  source 1..24: mol_type = other RNA
tcgccggatgatcggtactgacgc

SEQ ID NO: 75 - RNA, 24 nt, synthetic construct
  source 1..24: mol_type = other RNA
tctcgtcgccatcacaggacaggt

SEQ ID NO: 76 - DNA, 1959 bp, synthetic construct
  source 1..1959: mol_type = other DNA
atggcggcggcaacatcgtcttctccgatctccttaaccgctaaaccttcttccaaatcccctctacccatttccagattctcccttcccttctccttaaccccacagaaaccctcctcccgtctccaccgtcctctcgccatctccgccgttctcaactcacccgtcaatgtcgcacctgaaaaaaccgacaagatcaagactttcatctcccgctacgctcccgacgagccccgcaagggtgctgatatcctcgtggaagccctcgagcgtcaaggcgtcgaaaccgtcttcgcttatcccggaggtgcctccatggagatccaccaagccttgactcgctcctccaccatccgtaacgtcctcccccgtcacgaacaaggaggagtcttcgccgccgagggttacgctcgttcctccggcaaaccgggaatctgcatagccacttcgggtcccggagctaccaacctcgtcagcgggttagccgacgcgatgcttgacagtgttcctctcgtcgccatcacaggacaggtccctcgccggatgatcggtactgacgccttccaagagacgccaatcgttgaggtaacgaggtctattacgaaacataactatctggtgatggatgttgatgacatacctaggatcgttcaagaagcattctttctagctacttccggtagacccggaccggttttggttgatgttcctaaggatattcagcagcagcttgcgattcctaactgggatcaacctatgcgcttgcctggctacatgtctaggctgcctcagccaccggaagtttctcagttaggccagatcgttaggttgatctcggagtctaagaggcctgttttgtacgttggtggtggaagcttgaactcgagtgaagaactggggagatttgtcgagcttactgggatccctgttgcgagtacgttgatggggcttggctcttatccttgtaacgatgagttgtccctgcagatgcttggcatgcacgggactgtgtatgctaactacgctgtggagcatagtgatttgttgctggcgtttggtgttaggtttgataaccacgtcacgggaaagctcgaggcgtttgcgagcagggctaagattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttcccgttgagcttcaaaacgtttggagaagccattcctccgcagtacgcgattcaggtcctagacgagctaacccaagggaaggcaattatcagtactggtgttggacagcatcagatgtgggcggcgcagttttacaagtacaggaagccgaggcagtggctgtcgtcctcaggactcggagctatgggtttcggacttcctgctgcgattggagcgtctgtggcgaaccctgatgcgattgttgtggacattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacacctggaccgtacctgttggatgtcatctgtccgcaccaagaacatgtgttaccgatgatcccaagtggtggcactttcaaagatgtaataaccgaaggggatggtcgcactaagtactga

SEQ ID NO: 77 - protein, 652 aa, synthetic construct
MAAATSSSPISLTAKPSSKSPLPISRFSLPFSLTPQKPSSRLHRPLAISAVLNSPVNVAPEKTDKIKTFISRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDNHVTGKLEAFASRAKIVHIDIDSAEIGKNKTPHVSVCGDVKLALQGMNKVLENRAEELKLDFGVWRSELSEQKQKFPLSFKTFGEAIPPQYAIQVLDELTQGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQWEDRFYKANRAHTYLGDPARENEIFPNMLQFAGACGIPAARVTKKEELREAIQTMLDTPGPYLLDVICPHQEHVLPMIPSGGTFKDVITEGDGRTKY

(19) United States
(12) Patent Application Publication
(10) Pub. No.: US 2025/0369008 A1
(43) Pub. Date: Dec. 4, 2025

(54) DIVERSIFYING BASE EDITING

(71) Applicant: BASF Agricultural Solutions LLC, Durham, NC (US)

(72) Inventors: David DE VLEESSCHAUWER, Gent (BE); Frank MEULEWAETER, Gent (BE); Katelijn D'HALLUIN, Gent (BE)

(21) Appl. No.: 18/877,964

(22) PCT Filed: Jun. 23, 2023
(86) PCT No.: PCT/EP2023/067113

§ 371 Date: Dec. 20, 2024

(30) Foreign Application Priority Data
Jun. 23, 2022 (EP) 22180663.1

(51) Int. Cl. C12N 15/82; C12N 9/22; C12N 15/10; C12N 15/11

(52) U.S. Cl. CPC C12N 15/8274; C12N 9/226; C12N 15/1082; C12N 15/11; C12N 15/8213; C12Y 202/01006; C12N 2310/20

(57) ABSTRACT

The present invention relates to the field of increasing genetic diversity in a targeted way. In particular, it relates to the provision of methods and means for targeted sequence diversification using base editors with an expanded mutation spectrum, including the provision of Cas12a diversifying base editing systems, and uses thereof.

### TECHNICAL FIELD

The present invention relates to the field of increasing genetic diversity in a targeted way. In particular, it relates to the provision of methods and means for targeted sequence diversification using base editors with an expanded mutation spectrum, including the provision of Cas12a diversifying base editing systems, and uses thereof.

### BACKGROUND

The improvement of traits is an ongoing aim in agriculture and other fields. A classical approach to achieve this is random mutagenesis, usually via UV or EMS-induced mutagenesis. While these mutagenesis approaches allow the discovery of novel mutants, they are exceedingly time-consuming and labor intensive.

Moreover, these strategies are non-targeted and thus induce random mutations throughout the genome and, as such, do not allow directed evolution or manipulation of loci of interest without the simultaneous risk of causing undesired mutations in a genome of interest.

In contrast, targeted genetic modification can be achieved by CRISPR-Cas approaches. While these approaches do allow precise editing of genetic locations of interest, these are mostly limited to insertions and deletions and standard CRISPR-Cas approaches do not allow directed evolution. In order to enable directed evolution, strategies have been developed that rely on the in vitro creation of random or semi-random mutagenesis libraries. However, as these approaches are performed outside of the organisms of interest, they do not allow easy phenotypic analysis of the generated mutations.

With the creation of base editors, the CRISPR-Cas systems have been successfully modified to induce targeted point mutations instead of cleaving the target DNA. There are currently two predominant types: cytidine/cytosine (CBE) and adenine/adenosine base editors (ABE). CBEs are usually created by fusing a cytidine deaminase domain to a catalytically-impaired Cas9, either the dead (D10A/H840A) or a nickase (D10A) Cas9. A variety of cytidine deaminases have been used for base editing including APOBEC1 (A1), A3A, A3B, PmCDA1, AID, and their derivatives (Rees and Liu, 2018). CBEs catalyze the deamination of cytidines into uracil on the non-target DNA strand ultimately creating a C-G to T-A mutation (for CBEs, see Komor et al., 2016; Komor et al., 2017). Regarding the Cas9 variant suitable for base editors, nCas9 is thought to be more active than dCas9 because nicking of the target strand causes the non-target strand to be used as a template in mismatch mediated repair (e.g., Eid et al., 2018). Still, early base editors allow only a single type of conversion—C to T or A to G, respectively, and thus are not suitable in case a full range mutagenesis with high diversifying potential is of interest.

While recent development showed that these two types of base editors can be combined into so-called dual base editors, the resulting C to T and A to G conversions still offers only limited diversification. Therefore, there is a great need in the art for systems that allow diversification closer to random mutagenesis, while at the same time being targeted, i.e. inducing modifications specifically in a locus of interest, and allow the targeted diversification to be applied in situ, i.e. in the cell or organism of interest.

Another restriction is that base editing systems are currently limited to Cas9 base editors. While Cas12a, also called Cpf1, has—among other CRISPR-Cas systems—received increasing interest in recent years as an alternative to Cas9, Cas 12a base editor systems remain, however, mostly ineffectual, especially in plants. Moreover, no functional Cas12a dual base editing system has been described to date.

Therefore, it is the aim of the present invention to provide new and specifically optimized base editor systems, including Cas12a diversifying base editors, in order to allow in situ targeted diversification with an improved editing scope, but at the same time a high overall activity and base editing efficiency, which may be used for directed evolution approaches.

### SUMMARY OF THE INVENTION

In a first aspect, there is provided a method for targeted diversifying base editing of at least one target nucleic acid segment, the method comprising (a) providing at least one cell or construct comprising at least one target nucleic acid segment; (b) introducing into the target cell, or contacting with the target construct; (i) at least one diversifying base editor (DBE), or at least one nucleic acid molecule encoding the same; and (ii) at least one suitable guide RNA or at least one nucleic acid molecule encoding the same; (c) allowing complex formation of (i) the at least one diversifying base editor and (ii) the at least one suitable guide RNA; (d) obtaining at least one cell or construct comprising at least one modified target nucleic acid segment; wherein the total base editing efficiency of introducing at least one substitution of any kind into the at least on target nucleic acid segment is at least 0.2%, 0.5%, 1%, 5%, 10%, 15%, 20%, or at least 25%, wherein the upper limit is 100% or less; and/or wherein the rate of C to G substitutions is at least 0.1%, 0.5%, 1%, 5%, 10%, 15%, 20%, 30%, 40%, 50%, 60%, 70%, 80%, or at least 90% of the rate of C to T substitutions and/or the rate of C to A substitutions is at least 0.1%, 0.5%, 1%, 5%, 10%, 15%, 20%, 30%, 40%, 50%, 60%, 70%, 80%, or at least 90% of the rate of C to T substitutions; and/or wherein the at least one modification of the target nucleic acid segment occurs in an extended base editing window; wherein the method does not comprise treatment of the human or animal body by surgery or therapy and/or a diagnostic method practised on the human or animal body, and/or processes for modifying the germ line genetic identity of human beings.

In one embodiment of the first aspect, the diversifying base editor comprises a CRISPR-Cas portion originating from a Class 2 Type II CRISPR-Cas endonuclease, including a Cas9 endonuclease, or a Class 2 Type V CRISPR-Cas endonuclease, preferably wherein the diversifying base editor comprises a CRISPR-Cas portion originates from a Cas12a endonuclease.

In another embodiment of the first aspect, the at least one target cell is a prokaryotic cell, including a bacterial cell or an archaea cell, or a eukaryotic cell, including an insect cell, a mammalian cell or plant cell.

In another embodiment of the first aspect, the at least one target cell is a plant cell, including a plant protoplast.

In another embodiment of the first aspect, the at least one diversifying base editor comprises (i) one or more cytosine deaminase portion(s), (ii) one or more adenine deaminase portion(s), (iii) one or more CRISPR-Cas portion(s), preferably wherein the CRISPR-Cas domain does not cleave both strands of double-stranded DNA, (iv) one, two, three or more nuclear localization sequence(s); and (v) at least one linker region, preferably one or more linker region(s) between (i) and (ii), and optionally one or more linker regions between (ii) and (iii).

In another embodiment of the first aspect, the at least one diversifying base editor of step (b-i) is at least one diversifying base editor in form of a fusion protein, preferably wherein the portions (i), (ii) and (iii) as defined above are arranged, in N-terminal to C-terminal direction, in the order of (i)-(ii)-(iii) with one or more linker regions between each segment, further preferably wherein one, two, three or more nuclear localization sequence(s) (iv) are located at the C-terminus of the diversifying base editor, or wherein one or more nuclear localization sequence(s) (iii) is/are located at the N-terminus and one or more nuclear localization sequence(s) (iii) is/are located at the C-terminus of the diversifying base editor.

In another embodiment of the first aspect, the diversifying base editor comprises at least one further portion, preferably wherein the at least one further portion is selected from an ssDNA-, ssRNA-, or dsRNA-binding protein portion, including an MS2 protein portion, an affinity tag binding protein, a uracil glycosylase inhibitor portion and/or a uracil glycosylase portion, or any combination thereof.

In one embodiment, the at least one further portion comprises at least one uracil DNA N-glycosylase (UNG), optionally an Escherichia-coli-derived uracil DNA N-glycosylase (eUNG), optionally wherein the at least one UNG is delivered in trans with the at least one diversifying base editor of the present invention. For delivery of the at least one UNG, optionally the at least one eUNG, it may be desirable to express the at least one UNG or eUNG from a strong promoter, such as a 35S promoter (SEQ ID NO: 59). In preferred embodiments, the at least one UNG, optionally the at least one eUNG, is delivered in trans with the at least one diversifying base editor, wherein the at least one base editor is in form of a fusion protein as disclosed herein.

In another embodiment of the first aspect, the one or more adenine deaminase portion(s) and/or the one or more cytosine deaminase portion(s) is/are linked to at least one ssRNA- or dsRNA-binding protein portion, preferably at least one MS2 protein portion, and the at least one suitable guide RNA is adapted to allow interaction with the at least one ssRNA- or dsRNA-binding protein portion, preferably wherein the one or more adenine base editor portion and/or the one or more cytosine base editor portion is/are linked to at least one MS2 protein portion and the suitable guide RNA is adapted to comprise two MS2 stem-loops, optionally wherein the suitable guide RNA comprises a sequence selected from SEQ ID NO: 38 to SEQ ID NO: 41, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In another embodiment of the first aspect, the diversifying base editor comprises an amino acid molecule selected from any one of SEQ ID NO: 1-27, 52 or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the respective reference sequence.

In a second aspect, there is provided an edited cell, tissue, organ, material or whole organism obtained by or obtainable by a method according to the first aspect.

In a third aspect, there is provided a diversifying base editor, or a diversifying base editor complex additionally comprising at least one suitable guide RNA, or at least one nucleic acid molecule encoding the same, wherein the diversifying base editor is as defined in the first aspect.

In a fourth aspect, there is provided a vector or expression construct, or more than one vectors and expression constructs, each vector and/or expression construct comprising the at least one nucleic acid molecule of the third aspect, wherein different portions of the diversifying base editor are encoded on the same vector or expression construct or on different vectors or expression constructs, and/or wherein the diversifying base editor, or portions thereof, and the at least one suitable guide RNA are encoded on the same vector or expression construct or on different vectors or expression constructs.

In a fifth aspect, there is provided a cell comprising at least one diversifying base editor or at least one diversifying base editor complex, or at least one nucleic acid molecule encoding the same, of the third aspect; or at least one vector or expression construct of the fourth aspect; wherein the cell is a prokaryotic cell, including a bacterial cell or an archaea cell, or a eukaryotic cell, including an insect cell, a mammalian cell, including a human cell, or plant cell, including a plant protoplast, preferably wherein the cell is a plant cell, including a plant protoplast, optionally wherein the plant cell, including a plant protoplast, is a cell of, or originating from, a plant selected from wherein the at least one target cell is a plant cell of, or originating from, a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

In a sixth aspect, there is provided a kit comprising at least one diversifying base editor or at least one diversifying base editor complex, or at least one nucleic acid molecule encoding the same, of the third aspect; or at least one vector or expression construct of the fourth aspect; or at least one cell of the fifth aspect.

In a seventh aspect, there is provided a use of at least one diversifying base editor or at least one diversifying base editor complex, or at least one nucleic acid molecule encoding the same, of the third aspect; or at least one vector or expression construct of the fourth aspect; or at least one cell of the fifth aspect; or of at least one kit of the sixth aspect; for targeted directed evolution of at least one target nucleic acid segment, preferably in planta targeted directed evolution of at least one target nucleic acid segment, including a use for optimizing or modifying a trait in a plant, including the optimization or modification of a yield-related trait, or a disease or pathogen resistance related trait, wherein the disease is caused by, or the pathogen is selected from a virus, a bacterium, a fungus, a nematode, or an insect, or a herbicide-resistance related trait, or an abiotic-stress related trait, including a salinity or drought stress related trait, further including a use for identification of at least one lead gene.

### Definitions

The terms “adenine deaminase” and “adenosine deaminase” are used interchangeably herein. Likewise, the terms “cytidine deaminase” and “cytosine deaminase” are used interchangeably herein.

The term “base editor complex” as used herein refers to a complex of at least one base editor and at least one guide RNA suitable for at least one CRISPR-Cas portion of the at least one base editor. While the present invention includes base editors comprising more than one polypeptide, which form the diversifying base editor through non-covalent binding, these are also referred to as diversifying base editors or DBEs and are only referred to as base editor complexes if also comprising at least one suitable guide RNA. However, reference to a diversifying base editor or DBE without explicit reference to a complex, does not exclude that the base editor may be in a complex with at least one suitable guide RNA.

The term “base editing window” as used herein refers to that region usually in a genomic sequence, comprising a target nucleic acid segment to be modified, wherein the base editing window is that window where a diversifying base editor as guided by a suitable guide RNA is theoretically able to induce at least one targeted nucleotide exchange as base edit. This window is defined by the architecture of the diversifying base editor and the physical accessibility of the diversifying base editor as guided by a suitable guide RNA and the region, particularly a genomic region, to be modified.

A “diversifying base editor” or “DBE” as used herein refers to a to a base editor comprising at least one cytosine deaminase portion, at least one adenosine deaminase portion, at least one CRISPR-Cas portion, wherein the CRISPR-Cas portion may be modified to cleave only one strand of the target DNA or may be modified to not cleave any strand of the target DNA, and at least one nuclear localization sequence, wherein the DBE may further comprise one or more additional portions, such as an ssDNA, ssRNA, or dsRNA binding protein portion, a uracil glycosylase inhibitor portion and/or a uracil glycosylase portion, wherein the portions are covalently and/or non-covalently linked to each other, wherein non-covalent linking may also be achieved by covalent and/or non-covalent attachment of one or more portions that is/are not the CRISPR-Cas portion to a suitable guide RNA, which in turn interacts non-covalently with a CRISPR-Cas portion or a group of portions comprising a CRISPR-Cas portion, wherein covalent linking of portions may be achieved via at least one linker region.

The term “guide RNA” may refer to any RNA comprising a Cas-protein-binding region and a targeting region and is capable of guiding a Cas protein to a target nucleotide sequence being sufficiently complementary to the targeting region of the guide RNA as long as the target nucleotide sequence is located next to a Protospacer Adjacent Motif (PAM) suitable for the respective Cas protein. A “suitable guide RNA” as used herein refers to a guide RNA suitable for the CRISPR-Cas portion used as part of the DBE, i.e. a suitable guide RNA can bind to the employed CRISPR-Cas portion via the Cas-protein-binding region and the targeting region has complementarity to nucleotide sequence immediately upstream of a PAM sequence recognized by the employed CRISPR-Cas portion. As it is well known in the art, Cas12a systems typically rely on a single crRNA as guide RNA and Cas9 systems typically use a crRNA: tracrRNA duplex, which may be mimicked by a synthetic single guide RNA molecule. The skilled person is well aware of designing, expressing/synthesizing and adapting guide RNAs for the purposes needed.

“Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “|” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

“Indel” is a term for the random insertion or deletion of bases in the genome of an organism associated with the repair of a DSB by NHEJ. It is classified among small genetic variations, measuring from 1 to 10 000 base pairs in length. As used herein it refers to random insertion or deletion of bases in or in the close vicinity (e.g. less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

The term “material” as used herein when referring to material obtained or obtainable through methods of the present disclosure, refers to any material capable of comprising at least one target nucleic acid segment. “Material” may refer to cellular material as directly obtained or obtainable from an organism or group of organisms or as obtained or obtainable through lysis, solubilization and/or other means of preparation. Further, material may be self-proliferating, such as a reproductive system and/or seeds, or non-proliferating. Moreover, material may also refer to purified or synthetic material, such as plasmids, linear poly-or oligonucleotides, and the like.

The term “portion” as used herein refers to a functional unit of a diversifying base editor. A portion may be a single domain having one or more functionalities, such as an enzymatic activity or a binding activity, a portion may also consist of two or more domains that synergistically have a one or more functionalities. A portion may comprise or consist of a complete protein sequence of a given protein, such as a complete Cas12a protein sequence or a complete adenosine or cytidine deaminase protein sequence, or may comprise or consist of a part of the sequence of a polypeptide from which a portion originates in cases where it is known that a part of the sequence is sufficient to have the desired one or more functionalities. A portion may generally comprise or consist of a mutant amino acid sequence compared to the wild type protein sequence from which it originates.

The term “plant” as used herein encompasses whole plants, ancestors and progeny of the plants and plant parts, including seeds, shoots, stems, leaves, roots (including tubers), flowers, and tissues and organs. The term “plant” also encompasses plant cells, suspension cultures, callus tissue, embryos, meristematic regions, gametophytes, sporophytes, pollen and microspores.

The term “targeted diversifying base editing” as used herein refers to the state, quality, process and/or result of a diversifying base editor being guided to a target nucleic acid sequence by a suitable guide RNA through hybridization of the guide RNA and the target nucleic acid sequence, leading to base substitutions in an editing window at a target site. Targeted base editing does not preclude the existence of off-target base substitutions, i.e. base substitutions that do not occur at the target site. The skilled person is well aware of a variety of factors that influence off-target base substitutions.

A “target nucleic acid segment” as used herein refers to a stretch of DNA (single-stranded or double-stranded) or even RNA, such as genomic DNA for in vivo applications, and/or applications targeting cells in cell culture, or isolated DNA, e.g. plasmid DNA, for in vitro applications outside of living cells, in which the DBE-induced base substitutions occur, wherein either the target nucleic acid segment is within the target sequence (in applications in which the base editing window in smaller than the target sequence), or wherein the target sequence is within the target nucleic acid segment (in applications in which the base editing window extends beyond the target sequence). Generally, the “target nucleic acid segment” refers to the stretch of DNA that is within the target site and may extend, depending on the base editing window, up to 10 bp, up to 20 bp, up to 30 bp, or up to 40 bp next to the target site, including both directions.

A “target site” as used herein refers to both strands of a double-stranded DNA, i.e. a target strand—to which a guide RNA anneals—and a complementary non-target strand, wherein the target site is the stretch of DNA for with a guide RNA has suitable complementarity to at least one DNA strand.

“Total base editing efficiency” as used herein refers to the rate of introducing at least one nucleobase substitution within the target nucleic acid segment, i.e. at least one nucleobase in the target nucleic acid segment is substituted with a different nucleobase, irrespective of the type of substitution of a naturally occurring nucleobase against another. For example, a total base editing efficiency of 10% would mean that 10 out of 100 nucleic acid molecules carry at least one nucleobase substitution in the target nucleic acid segment as determined before and after, or without and with a DBE as disclosed herein. Typically, the total base editing efficiency is determined by sequencing, i.e. the percentage of reads showing a nucleobase substitution in the target nucleic acid segment relative to the total number of reads covering the target nucleic acid segment can be assumed to represent the total base editing efficiency within a reasonable margin of error for a given sequencing application.

### BRIEF DESCRIPTION OF FIGURES

FIG. 1A (FIG. 1A) shows the architecture of a Cas9-based DBE in comparison to a previous Cas9-base-editor (Li et al., 2020). Ubi denotes a maize Ubi-1 promoter, APOBEC3A denotes a human APOBEC3A deaminase, 48aa denotes an 48aa XTEN linker, 32aa denotes an 32aa XTEN linker, ecTadA and ecTadA7.10 denote a dimeric E. coli TadA/Tad7.10 deaminase, Npl denotes a nucleoplasmin NLS, UGI denotes a uracil glycosylase inhibitor, SV40 denotes a SV40 NLS, 3′35S denotes a Cauliflower mosaic virus 35S terminator, enCas9 denotes an “enhanced” Cas9 comprising the mutations K848A, K1003A and R1060A (in addition to D10A). The average base editing efficiency of the two base editors at the OsAAT locus is shown in FIG. 1B (FIG. 1B).

FIG. 2 (FIG. 2) shows different type of base substitutions in rice protoplast cells for an OsAAT locus by the different constructs shown in FIG. 1A. The y-Axis shows the number of identified substitutions within the target nucleic acid segment. Total read count amounts to 590176 and 659999 for STEME-1 and DBE-1, respectively.

FIG. 3 (FIG. 3) shows the total base editing efficiency of different LbCas 12a-DBE constructs in rice protoplasts for an OsAAT locus as determined by next-generation sequencing. The Y-axis shows the percentage of sequencing reads with base substitutions. The different used LbCas12a-DBE architectures are (all dpNLS portions in the shown constructs have the sequence of SEQ ID NO: 49):
1: hA3A-48aa-XTEN-linker-ecTadA-32aa-XTEN-linker-TadA7.10-32aa-XTEN-linker-enLbCas 12a(D832A)-SV40-NLS(3x) (SEQ ID NO: 17)
2: hA3A-48aa-XTEN-linker-ecTadA-32aa-XTEN-linker-TadA7.10-32aa-XTEN-linker-enLbCas12a(D832A/R1138A)-SV40-NLS(3x) (corresponding to SEQ ID NO: 17 with an additional R1138A mutation in the enLbCas12a)
3: hA3A-48aa-XTEN-linker-ecTadA-32aa-XTEN-linker-TadA7.10-32aa-XTEN-linker-enLbCas12a(D832A/E795L)-SV40-NLS(3x) (SEQ ID NO: 21)
4: hA3A (R128A)-48aa-XTEN-linker-ecTadA-32aa-XTEN-linker-TadA8e(V106W)-32aa-XTEN-linker-enLbCas12a(D832A)-SV40-NLS(3x) (SEQ ID NO: 20 )
5: hA3A (R128A)-48aa-XTEN-linker-ecTadA-32aa-XTEN-linker-TadA8e(V106W)-32aa-XTEN-linker-enLbCas12a(D832A/R1138A)-SV40-NLS(3x) (corresponding to SEQ ID NO: 20 with an additional R1138A mutation in the enLbCas12a)
6: hA3A (R128A)-48aa-XTEN-linker-ecTadA-32aa-XTEN-linker-TadA8e(V106W)-32aa-XTEN-linker-enLbCas12a(D832A/E795L)-SV40-NLS(3x) (corresponding to SEQ ID NO: 20 with an additional E795L mutation in the enLbCas 12a)
7: dpNLS (dual portion nuclear localization signal)-hA3A-48aa-XTEN-linker-monomeric TadA8e-32aa-XTEN-linker-LbCas12a(D156R/D832A)-dpNLS (SEQ ID NO: 7)
8: dpNLS-hA3A-48aa-XTEN-linker-monomeric TadA8e-32aa-XTEN-linker-LbCas12a(D156R/D832A/K932G/N933G)-dpNLS (SEQ ID NO: 8)
9: dpNLS-hA3A-48aa-XTEN-linker-monomeric TadA8e-32aa-XTEN-linker-LbCas12a(D156R/D832A/E795L)-dpNLS (SEQ ID NO: 9)
10: dpNLS-hA3A-48aa-XTEN-linker-monomeric TadA8e-GGGGS-linker-(5x)-LbCas12a(D156R/D832A)-dpNLS (SEQ ID NO: 10)

FIG. 4A and 4B (FIG. 4A and FIG. 4B) show the results of base editing with LbCas12a-DBE-10 (SEQ ID NO: 10) in oilseed rape (Brassica napus) and soybean (Glycine max) protoplasts. FIG. 4A shows successful editing of an extrachromosomal mBFP or dGFP reporter in Brassica napus. 35S>eGFP denotes a positive control in which cells are transformed with eGFP under control of a 35S promoter. R denotes the red channel and G denotes the green (GFP) channel. The average base editing efficiency of DBE-10 at the FAD2 and ALS3 loci in Brassica napus and at the FAD2 locus in Glycine max as determined by next-generation sequencing or digital droplet PCR analysis is shown in FIG. 4B.

FIG. 5A and 5B (FIG. 5A and FIG. 5B) show four different Cas12a guide RNA architectures bearing two MS2 stem-loops. Gray shading marks the MS2 stem-loops. crRNA1: SEQ ID NO: 38, crRNA2: SEQ ID NO: 39, crRNA3: SEQ ID NO: 40, crRNA4: SEQ ID NO: 41. The MS2-tagging system is disclosed in Beach DL, Keene JD. Methods Mol. Biol. 2008;419:69-91.

FIG. 6 (FIG. 6) shows the results of an in vitro digest of a double-stranded PCR product with the four different Cas 12a-MS2 guide RNAs shown in FIG. 5. crRNA-AAT denotes a control guide RNA without MS2 stem-loops, crtl denotes a negative control without a guide RNA. U denotes uncleaved DNA, C denotes cleaved DNA.

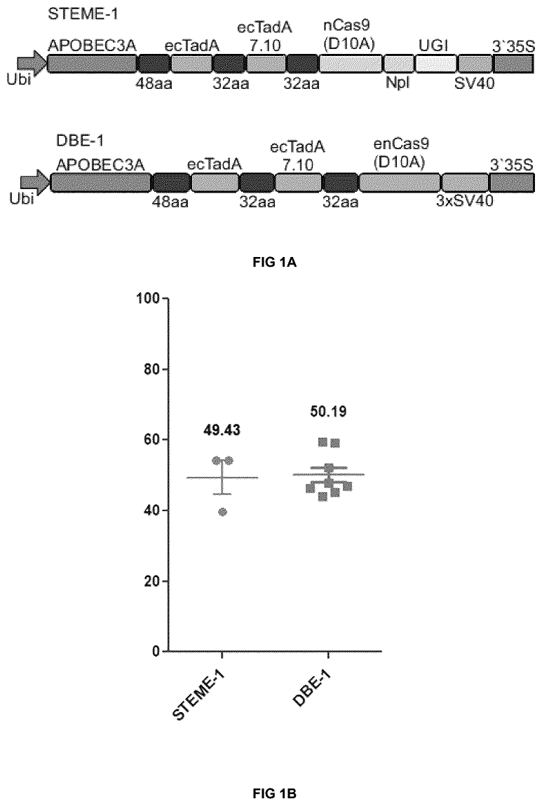

FIG. 7 (FIG. 7) shows the total base editing efficiency in rice protoplasts for an OsAAT locus of dLbCas12a-directed MS2-hA3A fusions with the four different Cas12a-MS2 guide RNAs shown in FIG. 5. The Y-axis shows the percentage of sequencing reads with base substitutions. nCas9-DBE1 denotes the Cas9 DBE-1 editor shown in FIG. 1, Cas12a-DBE-10 refers to construct 10 in FIG. 3 (SEQ ID NO: 10).

FIG. 8 (FIG. 8) displays cleavage activities of the different Cas12a gRNAs shown in Table 4 as determined by next-generation sequencing. The Y axis shows the percentage of NGS reads with indels. Black and white bars represent data from two independent experiments.

FIG. 9A (FIG. 9A) shows a stereoview of the bispyribac binding sites in AtAHAS (source: Garcia et al., 2017). The herbicide is shown in a ball and stick model, whereas key residues for herbicide binding are depicted as stick models. The ‘ denotes that these residues are from the neighboring subunit. Binding site residues with identified mutations are encircled. FIG. 9B (FIG. 9B) then shows the Connolly surface and herbicide blocking the substrate access channel in AtAHAS. Bispyribac is represented in a ball and stick model. Binding site residues with identified mutations are encircled.

### DETAILED DESCRIPTION

To achieve the aim of the present invention, a novel class of Cas9-based diversifying base editors enabling a broader mutation spectrum has been developed. Moreover, Cas 12a-based diversifying base editors have been de novo designed and optimized.

In a first aspect there may be provided a method for targeted diversifying base editing of at least one target nucleic acid segment, comprising (a) providing at least one cell or construct comprising at least one target nucleic acid segment; (b) introducing into the target cell, or contacting with the target construct; (i) at least one diversifying base editor (DBE), or at least one nucleic acid molecule encoding the same; and (ii) at least one suitable guide RNA or at least one nucleic acid molecule encoding the same; (c) allowing complex formation of (i) the at least one diversifying base editor and (ii) the at least one suitable guide RNA; (d) obtaining at least one cell or construct comprising at least and modified target nucleic acid segment;
wherein the total base editing efficiency of introducing at least one substitution of any kind into the at least on target nucleic acid segment is at least 0.2%, 0.5%, 1%, 5%, 10%, 15%, 20%, or at least 25%, wherein the upper limit is 100% or less; or
wherein the rate of C to G substitutions is at least 0.1, 0.5%, 1%, 5%, 10%, 15%, 20%, 30%, 40%, 50%, 60%, 70%, 80%, or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; or
wherein the rate of C to A substitutions is at least 0.1%, 0.5%, 1%, 5%, 10%, 15%, 20%, 30%, 40%, 50%, 60%, 70%, 80%, or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; or
wherein the at least one modification of the target nucleic acid segment occurs in an extended base editing window; or
wherein the total base editing efficiency of introducing at least one substitution of any kind into the at least on target nucleic acid segment is at least 0.2%, 0.5%, 1%, 5%, 10%, 15%, 20%, or at least 25%, wherein the upper limit is 100% or less; and wherein the rate of C to G substitutions is at least 0.1, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; or
wherein the total base editing efficiency of introducing at least one substitution of any kind into the at least on target nucleic acid segment is at least 0.2%, 0.5%, 1%, 5%, 10%, 15%, 20%, or at least 25%, wherein the upper limit is 100% or less; and wherein the rate of C to G substitutions is at least 0.1, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; and wherein the rate of C to A substitutions is at least 0.1%, 0.5%, 1%, 5%, 10%, 15%, 20%, 30%, 40%, 50%, 60%, 70%, 80%, or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; or
wherein the total base editing efficiency of introducing at least one substitution of any kind into the at least on target nucleic acid segment is at least 0.2%, 0.5%, 1%, 5%, 10%, 15%, 20% or at least 25%, wherein the upper limit is 100% or less; and wherein the rate of C to G substitutions is at least 0.1, 0.5%, 1%, 5%, 10%, 15%, 20%, 30%, 40%, 50%, 60%, 70%, 80%, or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; and wherein the at least one modification of the target nucleic acid segment occurs in an extended base editing window, or
wherein the total base editing efficiency of introducing at least one substitution of any kind into the at least on target nucleic acid segment is at least 0.2%, 0.5%, 1%, 5%, 10%, 15%, 20% or at least 25%, wherein the upper limit is 100% or less; and wherein the rate of C to A substitutions is at least 0.1%, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; or
wherein the total base editing efficiency of introducing at least one substitution of any kind into the at least on target nucleic acid segment is at least 0.2%, 0.5%, 1%, 5%, 10%, 15%, 20% or at least 25%, wherein the upper limit is 100% or less; and wherein the rate of C to A substitutions is at least 0.1%, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; and wherein the at least one modification of the target nucleic acid segment occurs in an extended base editing window, or
wherein the total base editing efficiency of introducing at least one substitution of any kind into the at least on target nucleic acid segment is at least 0.2%, 0.5%, 1%, 5%, 10%, 15%, 20%, or at least 25%, wherein the upper limit is 100% or less; and wherein the at least one modification of the target nucleic acid segment occurs in an extended base editing window, or
wherein the rate of C to G substitutions is at least 0.1, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; and wherein the rate of C to A substitutions is at least 0.1%, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; or
wherein the rate of C to G substitutions is at least 0.1, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; and wherein the at least one modification of the target nucleic acid segment occurs in an extended base editing window, or
wherein the rate of C to G substitutions is at least 0.1, 0.5%, 1%, 5%, 10%, 15%, 20%, 30%, 40%, 50%, 60%, 70%, 80% or at least 90% of the rate of C to T substitutions, optionally wherein the upper limit 100% or less, 110% or less, 120% or less, 130% or less, 140% or less, 150% or less, 160% or less, 170% or less, 180% or less, 190% or less, or 200% or less; and wherein the rate of C to A substitutions is at least 0.1%, 0.5%, 1%, 5%, 10%, 15%, 20%, 30%, 40%, 50%, 60%, 70%, 80%, or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; and wherein the at least one modification of the target nucleic acid segment occurs in an extended base editing window, or wherein the rate of C to G substitutions is at least 0.1, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; and wherein the at least one modification of the target nucleic acid segment occurs in an extended base editing window, or wherein the rate of C to A substitutions is at least 0.1%, 0.5%, 1%, 5%, 10%, 15%, 20%, 30%, 40%, 50%, 60%, 70%, 80% or at least 90% of the rate of C to T substitutions, optionally wherein the upper limit is 100% or less, 110% or less, 120% or less, 130% or less, 140% or less, 150% or less, 160% or less, 170% or less, 180% or less, 190% or less, or 200% or less; and wherein the at least one modification of the target nucleic acid segment occurs in an extended base editing window, preferably wherein the diversifying base editor comprises a CRISPR-Cas portion originating from a Class 2 Type V CRISPR-Cas endonuclease, wherein the Class 2 Type V CRISPR-Cas endonuclease may be a Cas12a endonuclease, or portion thereof, wherein the method does not comprise treatment of the human or animal body by surgery or therapy and/or a diagnostic method practised on the human or animal body, and/or processes for modifying the germ line genetic identity of human beings.

In some embodiments, the method is performed outside of living cells, wherein the at least one target nucleic acid segment is comprised in at least one construct, such as at a linear DNA molecule, e.g. a PCR product or a restriction digest product, or a DNA vector, including a plasmid vector. In such embodiments, the at least one DBE is typically used in a purified form. The skilled person is well aware of a variety of standard procedures for Protein expression and purification. The at least one guide RNA may be purified from e.g. in vitro transcription or de-novo synthesized.

In other embodiments the method is performed within living cells, i.e. the at least one DBE, the at least one suitable guide RNA, or the at least one nucleic acid encoding the same, are introduced into the at least one cell. The at least one DBE and the at least one suitable guide RNA may be introduced separately, together, and/or as an RNP complex. In embodiments relating to the introduction of at least one nucleic acid molecule encoding the at least one DBE and the at least one suitable guide RNA, a DBE may be encoded on the same nucleic acid molecule as the at least one suitable guide RNA, or it may be encoded on a different nucleic acid molecule. The nucleic acid molecule may be RNA, typically an mRNA molecule, or DNA, including DNA expression vectors, including expression plasmid vectors. The at least one guide RNA is typically provided either directly as a guide RNA molecule or as DNA encoding the same. The skilled person is well aware of the design and preparation of different nucleic acid molecules, as well as various different methods of introducing proteins, nucleic acids and RNPs into living cells.

In certain embodiments the total base editing efficiency of introducing at least one substitution of any kind into the at least on target nucleic acid segment is 30% to 100% or 35% to 100%, or 40% to 100% or 45% to 100% or 50% to 100%.

A “modified target nucleic acid segment” as used herein refers to the presence of at least one nucleobase substitutions of any kind within the target nucleic acid segment, wherein—unless otherwise specified—a substitution of any kind refers to a substitution of any of the four natural nucleobases A, C, G or T to any different of the four natural nucleobases.

The at least one nucleic acid molecule encoding the at least one DBE according to the various embodiments and aspects herein, may be codon optimized and may further comprise a nucleic acid sequence encoding at least one compatible guide RNA. In any of the embodiments described herein, a nucleic acid sequence or molecule may be operatively linked to a variety of promoters and other regulatory elements for expression in a cell and/or organism of interest.

The methods according to the embodiments and aspects may comprise the additional step of regenerating at least one population of edited cells, tissues, organs, materials or whole organisms from the at least one edited cell or construct.

In one embodiment of the first aspect, the diversifying base editor comprises a CRISPR-Cas portion originating from a naturally occurring and later of artificially modified Class 2 Type II CRISPR-Cas endonuclease, including a Cas9 endonuclease, or a Class 2 Type V CRISPR-Cas endonuclease, preferably wherein the diversifying base editor comprises a CRISPR-Cas portion originates from a Cas12a endonuclease.

The CRISPR-Cas portion may comprise or consist of a mutant Cas9 or Cas 12a amino acid sequence. Typically, the CRISPR-Cas portion comprises at least one mutation causing the CRISPR-Cas portion to not cleave both strands of a double-stranded DNA, thereby turning the CRISPR-Cas portion into a nickase (cleaving one strand of a double-stranded DNA) or dead (not cleaving DNA) CRISPR-Cas portion. The CRISPR-Cas portion may comprise further mutations altering PAM-specificity, thermotolerance and/or other characteristics.

In a preferred embodiment using a CRISPR-Cas9 portion, the CRISPR-Cas portion comprises or consists of an SpCas9 having the mutations D10A, K848A, K1003A, and R1060A, referred to as “enCas9” or “enCas9 nickase” herein. The K848A, K1003A, R1060A mutations have been shown to weaken non-target strand binding by neutralizing positively charged residues in the non-target strand groove, thus promoting dissociation of nCas9 from DNA after nicking the target locus (Slaymaker et al., 2016).

Preferred CRISPR-Cas12a portions comprise or consist of an LbCas12a having the mutations D156R and D832A, optionally further having the double mutation G532R/K538R, and/or the mutation E795L. In one embodiment the CRISPR-Cas portions comprises or consists of LbCas12a-D156R/G532R. In another embodiment, the CRISPR-Cas-portion comprises or consists of LbCas12a-D156R/G532R/K538R/D832A. In a further embodiment, the CRISPR-Cas portion comprises or consists of LbCas12a-D156R/D832A/E795L. In yet another embodiment, the CRISPR-Cas-portion comprises or consists of D156R/G532R/K538R/D832A/E795L.

In one embodiment of the first aspect, the at least one target cell is a prokaryotic cell, including a bacterial cell or an archaea cell, or a eukaryotic cell, including an insect cell, a mammalian cell or plant cell.

In another embodiment according to the first aspect, the at least one target cell is a plant cell, including a plant protoplast, optionally wherein the plant cell, including the plant protoplast, is a cell of, or originating from, a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

Preferred plants are Abelmoschus spp., Allium spp., Apium graveolens, Asparagus officinalis, Avena spp. (e.g. Avena sativa, Avena fatua, Avena byzantina, Avena fatua var. sativa, Avena hybrida), Beta vulgaris, Brassica spp. (e.g. Brassica napus, Brassica rapa ssp. [canola, oilseed rape, turnip rape]), Capsicum spp., Citrullus lanatus, Cucumis spp., Cynara spp., Daucus carota, Glycine spp. (e.g. Glycine max, Soja hispida or Soja max), Gossypium hirsutum, Helianthus spp. (e.g. Helianthus annuus), Hordeum spp. (e.g. Hordeum vulgare), Lactuca sativa, Medicago sativa, Oryza spp. (e.g. Oryza sativa, Oryza latifolia), Pennisetum sp., Saccharum spp., Secale cereale, Solanum spp. (e.g. Solanum tuberosum, Solanum integrifolium or Solanum lycopersicum), Sorghum bicolor, Spinacia spp., Triticum spp. (e.g. Triticum aestivum, Triticum durum, Triticum turgidum, Triticum hybernum, Triticum macha, Triticum sativum, Triticum monococcum or Triticum vulgare), or Zea mays.

Preferred plants, in certain embodiments, may also be selected from Brassica spp. (e.g. Brassica napus, Brassica rapa ssp. [canola, oilseed rape, turnip rape]), Capsicum spp., Glycine spp. (e.g. Glycine max, Soja hispida or Soja max), Gossypium hirsutum, Helianthus spp. (e.g. Helianthus annuus), Oryza spp. (e.g. Oryza sativa, Oryza latifolia), Solanum spp. (e.g. Solanum tuberosum, Solanum integrifolium or Solanum lycopersicum), Triticum spp. (e.g. Triticum aestivum, Triticum durum, Triticum turgidum, Triticum hybernum, Triticum macha, Triticum sativum, Triticum monococcum or Triticum vulgare), or Zea mays.

In another embodiment of the first aspect, the at least one diversifying base editor of step (b-i) comprises (i) one or more cytosine deaminase portion(s), (ii) one or more adenine deaminase portion(s), (iii) one or more CRISPR-Cas portion(s), preferably wherein the CRISPR-Cas domain does not cleave both strands of double-stranded DNA, (iv) one, two, three or more nuclear localization sequence(s); and (v) at least one linker region, preferably one or more linker region(s) between (i) and (ii), and optionally one or more linker regions between (ii) and (iii).

A variety of adenine and cytosine deaminases are known to the skilled person (e.g. Fan et al., 2021; Jeong et al., 2020; Yan et al., 2021). Any adenine deaminase and/or cytosine deaminase, including variants of known deaminases may be used in a diversifying base editor of the present invention, if combined in a suitable way with the other building blocks following the construction details as disclosed herein.

In some embodiments, a cytosine deaminase may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, an activation induced deaminase (AID), such as hAID or AICDA, rAPOBEC1, an PpAPOBEC1, an AmAPOBEC1, an SsAPOBEC3B, an RrA3F, a FERNY, a cytosine deaminase, such as CDA1, CDA2, pmCDA1, or atCDA1, or a cytosine deaminase acting on rRNA (CDAT), or a variant thereof.

In preferred embodiments, the one or more cytosine deaminase portion(s) comprise(s) or consist(s) of a human apolipoprotein B mRNA editing enzyme catalytic polypeptide-like 3A (hA3A). In one embodiment, the one or more cytosine deaminase portions comprise or consist of a hA3A having the mutation R128A, or the mutation Y130F or the double mutation W104A/P134Y.

An adenosine deaminase portion may comprise or consist of a monomeric adenosine deaminase or a dimeric adenosine deaminase, wherein the monomers of a dimeric adenosine deaminase are preferably linked via at least one linker region, preferably via a 32aa XTEN linker.

In some embodiments, an adenine deaminase portion may be a tRNA-specific adenosine deaminase, such as TadA (Gaudelli et al., 2017), or an adenosine deaminase 1 (ADA1), ADA2; an adenosine deaminase acting on RNA 1 (ADAR1), ADAR2, ADAR3 (e.g., Savva et al., 2012); or an adenosine deaminase acting on tRNA 1 (ADAT1), ADAT2, ADAT3, or variant thereof.

In some embodiments, a TadA may be from E. coli (ecTadA). In some embodiments, the TadA may be modified and/or truncated. In certain embodiments, a TadA does not comprise an N-terminal methionine. TadA deaminases that may be used as part of a base editor or base editor complex according to the present invention may for example be a TadA8, TadA8e, TadA8 , TadA7.9 TadA7.10, TadA7.10d, TadA8.17, TadA8.20, TadA9, or a variant thereof.

In preferred embodiments, the one or more adenosine deaminase portion(s) comprise(s) or consist(s) of a dimeric ecTadA/ecTadA7.10, or a dimeric ecTadA/TadA8e-V106W, or a monomeric TadA8e or a monomeric TadA9, preferably the one or more adenosine deaminase portion(s) comprise(s) or consist(s) of a monomeric TadA9.

In one embodiment the DBE, comprises at least one monopartite or a bipartite nuclear localization signal (NLS), preferably at least one NLS comprising or consisting of the sequence of SEQ ID NO:49. Any other NLS, and combination of NLSs, specifically tested for a DBE core structure as disclosed herein, or combinations thereof, can also be used. Suitable NLSs are disclosed, for example, in Lange et al., 2010.

The terms “nuclear localization signal”, “nuclear localization sequence” and “NLS” are used interchangeably herein.

In certain embodiments, at least two or at least three, for example, three repeats of an SV40 NLS, may be used.

In certain embodiments, a dual portion NLS (dpNLS), at least one at the C- or N-terminus of the DBE and at another position within the DBE, preferably at the C-terminus and at the N-Terminus, may be used. At least one, or both, of the portions of the dpNLS may be a bipartite NLS, for example, the sequence of SEQ ID NO:49, or a sequence having at least 99% identity thereto. In certain embodiments, only one of the portions of the dpNLS will be a bipartite NLS, including SEQ ID NO:49, or a sequence having at least 99% identity thereto, and the second portion will be, for example, a triple SV40 NLS as disclosed herein.

In a preferred embodiment, the DBE comprises an NLS comprising or consisting of the sequence of SEQ ID NO: 49, or a sequence having at least 99% identity thereto, at the N-terminus and/or at the C-terminus, preferably at the N-terminus and at the C-terminus.

In all embodiments using non-covalent linking of portions to form the DBE, each polypeptide that forms part of the DBE preferably comprises as least one NLS, more preferably wherein each polypeptide that forms part of the DBE comprises an SV40 NLS, preferably three repeats of an SV40 NLS, or, more preferably, a dual portion NLS (dpNLS); at the C-terminus and/or the N-terminus and at a second location within the DBE, preferably at the C-terminus and at the N-Terminus, preferably, wherein at least one, or even both, of the dpNLS sequences is SEQ ID NO:49, or a sequence having at least 99% identity thereto.

Non-covalent binding may be achieved by any binding pair, such as affinity tags, biotin-streptavidin interaction or e.g. FRB-FKBP (Inobe and Nukina, 2016), allowing a specific interaction. Non-covalent binding may be achieved by non-covalent protein-protein interaction and/or by non-covalent protein-RNA interaction with the guide RNA. If the DBE is formed by non-covalent association with the guide RNA, the binding pair may be an RNA-binding portion fused to a part of the DBE and a modification of the guide RNA, such as the inclusion a stem-loop and/or a binding sequence allowing specific interaction with said RNA-binding portion. Thereby, any portion or group of portions may be non-covalently linked—via the guide RNA—to the CRISPR-Cas portion or group of portions comprising the CRISPR-Cas portion.

In certain embodiments using non-covalent linking, the DBE comprises or consists of a first group of portions covalently linked to each other, wherein one portion may be fused to another portion via at least one linker region, and a second group of portions covalently linked to each other, wherein one portion may be fused to another portion via at least one linker region, wherein the first and the second group of portions each comprise a portion that allows non-covalent linking of the first group of portions to the second group of portions.

In some embodiments of non-covalent linking, the first group of portions comprises the CRISPR-Cas portion and the second group of portions comprises an ssRNA-and/or an dsRNA-binding portion, and the suitable guide RNA is modified to allow binding to the ssRNA-and/or an dsRNA-binding portion.

In certain embodiments of non-covalent linking, a first group of portions comprises or consists of one or more CRISPR-Cas portions, optionally one or more further portions, such as a uracil glycosylase inhibitor portion, a uracil glycosylase portion and/or an ssDNA-binding portions, and one, two, three or more nuclear localization signals at the C and/or N-terminus of the first group of portions, wherein one portion may be fused to another portion via at least one linker region; and wherein a second group of portions comprises or consists of one or more adenosine deaminase portions, and/or one or more cytosine deaminase portions and one or more ssRNA-and/or an dsRNA-binding portions, preferably an MS2 protein portion, and one, two, three or more nuclear localization signals at the C and/or N-terminus of the second group of portions, wherein one portion may be fused to another portion via at least one linker region.

A linker region as used herein refers to a polypeptide linker, wherein a fist portion is fused to the N-terminus of the polypeptide linker and a second portion is fused to the C-terminus of the polypeptide linker. There is a variety of available polypeptide linker regions recognized and used in the art.

A polypeptide linker may be a GS linker, such as a polypeptide linker comprising or consisting of an amino acid sequence of (GGS)n, S(GGS)n, or SGGS, wherein n is a number of 1-20 (e.g., 1, 2, 3, 4, 5, 6, 7, 8, 9, 10, 11, 12, 13, 14, 15, 16, 17, 18, 19, or 20). A polypeptide linker may also comprise consist of the amino acid sequence: SEQ ID NO: 45. A polypeptide linker may also comprise or consist of the amino acid sequence: SEQ ID NO: 46, also referred to as XTEN linker. Further, a polypeptide linker may comprise or consist of the amino acid sequence: SEQ ID NO: 47, which is also called GS-XTEN-GS linker and is referred to “32aa XTEN linker” herein. Moreover, a polypeptide linker may comprise or consist of the amino acid sequence SEQ ID NO: 48, referred to as “48aa XTEN linker” herein.

In one embodiment, the one or more linker region(s) between portions (i) and (ii) as defined above, comprise or consist of an 48aa XTEN linker.

In one embodiment, the one or more linker region(s) between portions (ii) and (iii) as defined above, comprise or consist of an 32aa XTEN linker or, preferably, a GS linker consisting of three, five or six repeats of a of the amino acid sequence GGGGS (cf. SEQ ID NO: 51) ((GGGGS)₃, (GGGGS)₅ and (GGGGS)₆, respectively). In another embodiment, the linker between portion (ii) and portion (iii) is replaced by a non-sequence-specific ssDNA-binding portion, preferably a Rad51 ssDNA-binding domain (Rad51ssDBD), or a non-sequence-specific ssDNA-binding portion, preferably a Rad51ssDBD, is added to the linker region, preferably to a (GGGGS)₅ linker region, between portion (ii) and portion (iii). In yet another embodiment, the linker between portion (ii) and portion (iii) is replaced by non-covalent linking as described above.

In a preferred embodiment, the linker between portion (ii) and (iii) as defined above comprises or consists of a (GGGGS)₅ linker.

In certain embodiments, different portions may be linked via bioconjugation, for example using the SNAP tag system (Hussain et al., 2013); the Halo tag system (Los et al., 2008); the CLIP tag system (Gautier et al., 2008) or any joining of specific biomolecules collectively referred to as “click chemistry” in the art.

In another embodiment of the first aspect, the at least one diversifying base editor of step (b-i) is at least one diversifying base editor in form of a fusion protein, preferably wherein the portions (i), (ii) and (iii) as defined above are arranged, in N-terminal to C-terminal direction, in the order of (i)-(ii)-(iii) with one or more linker regions between each segment, further preferably wherein one, two, three or more nuclear localization sequence(s) (iv) are located at the C-terminus of the diversifying base editor, or wherein one or more nuclear localization sequence(s) (iii) is/are located at the N-terminus and one or more nuclear localization sequence(s) (iii) is/are located at the C-terminus of the diversifying base editor.

In one embodiment, three repeats of an SV40 NLS are located at the C-terminus of the DBE.

In another embodiment, a dual portion (dpNLS) is fused to the N-terminus or the C-terminus, preferably at the N-terminus and the C-terminus of a DBE as disclosed herein, preferably wherein at least one of the dpNLS sequences is SEQ ID NO: 49, or a sequence having at least 99% identity thereto. In other embodiments, both parts of the dpNLS have a sequence of SEQ OD NO:49, or a sequence having at least 99% identity thereto.

In another embodiment of the first aspect, the diversifying base editor comprises at least one further portion, preferably wherein the at least one further portion is selected from an ssDNA-, ssRNA-, or dsRNA-binding protein portion, including an MS2 protein portion, an affinity tag binding protein, a uracil glycosylase inhibitor portion and or a uracil glycosylase portion, or any combination thereof.

In one embodiment, the at least one further portion comprises or consists of a uracil glycosylase inhibitor (UGI).

In one embodiment, the DBE comprises uracil DNA glycosylase (UDG), including a uracil-n-glycosylase (UNG).

In certain embodiments, the DBE does not comprise a uracil glycosylase inhibitor portion and/or does not comprise a uracil glycosylase portion.

In certain embodiments, the DBE comprises a non-specific ssDNA-binding portion, preferably an ssDNA-binding domain of Rad51 (Rad51ssDBD). For Cas9 cytosine base editors, it has been previously shown that a RAD51ssDBD between the Cas9 and the cytosine deaminase may increase base editing efficiency and extend the base editing window in cell lines an mouse embryos (Zhang et al., 2020). In certain embodiments, the Rad51ssDBD is used instead of a linker region between portion (ii) and portion (iii), or a Rad51ssDBD is added to the linker region, preferably to a (GGGGS)₅ linker region, between portion (ii) and portion (iii).

In another embodiment of the first aspect, the one or more adenine deaminase portion(s) and/or the one or more cytosine deaminase portion(s) is/are linked to at least one ssRNA- or dsRNA-binding protein portion, preferably at least one MS2 protein portion, and the at least one suitable guide RNA is adapted to allow interaction with the at least one ssRNA- or dsRNA-binding protein portion, preferably wherein the one or more adenine base editor portion and/or the one or more cytosine base editor portion is/are linked to at least one MS2 protein portion and the suitable guide RNA is adapted to comprise two MS2 stem-loops.

MS2-tagging strategies rely on the binding of the MS2 bacteriophage coat protein (referred to as “MS2 protein” or, in the context of a DBE, a “MS2 protein portion” herein) to a hairpin structure from the phage genome referred to as “MS2 (stem-)loop” herein.

In one embodiment, the one or more CRISPR-Cas portion(s) is/are one or more Cas12a portion(s), and the one or more adenine deaminase portion(s) and/or the one or more cytosine deaminase portion(s) is/are linked to one or more MS2 protein portion(s), and the guide RNA comprises two MS2 loops, optionally wherein the guide RNA comprises a sequence of SEQ ID NO: 38, or SEQ ID NO: 39, or SEQ ID NO: 40, or SEQ ID NO: 41, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In certain embodiments, the (ii) one or more adenosine deaminase portion(s) is/are fused to one or more MS2 protein portions and to one or more NLS portions, optionally as a fusion protein having the amino acid sequence of SEQ ID NO: 42 or a sequence having at least 90%, 91%, 92%, 93%, 94%, 95%, 96%, 97%, 98% or at least 99% sequence identity thereto; and the (i) one or more cytosine deaminase portion(s) is/are fused to portions (iii) and (iv), as a second fusion protein, preferably with one or more linker regions, optionally a 32aa-XTEN-linker, a 48aa-XTEN-linker, a (GGGGS)₅ or a (GGGGS)₆ linker, between (i) and (iii).

In certain embodiments, the (i) one or more cytosine deaminase portion(s) is/are fused to one or more MS2 protein portions and to one or more NLS portions, optionally as a fusion protein having the amino acid sequence of SEQ ID NO: 43 or SEQ ID NO: 44 or a sequence having at least 90%, 91%, 92%, 93%, 94%, 95%, 96%, 97%, 98% or at least 99% sequence identity thereto; and the (ii) one or more adenosine deaminase portion(s) is/are fused to portions (iii) and (iv), preferably with one or more linker regions between (ii) and (iii).

In certain embodiments, the (i) one or more cytosine deaminase portion(s) is/are fused to the (ii) one or more adenosine deaminase portion(s), preferably via one or more linker regions, to one or more MS2 protein portions, and to one or more NLS portions; and the (iii) one or more CRISPR-Cas portions is/are fused to portion (iv) as a second fusion protein.

In certain embodiments, the (i) one or more cytosine deaminase portion(s) is/are fused to one or more MS2 protein portions and to one or more NLS portions, optionally as a fusion protein having the amino acid sequence of SEQ ID NO: 43 or SEQ ID NO: 44 or a sequence having at least 90%, 91%, 92%, 93%, 94%, 95%, 96%, 97%, 98% or at least 99% sequence identity thereto, and the (ii) one or more adenosine deaminase portion(s) is/are fused to one or more MS2 protein portions and to one or more NLS portions, optionally as a fusion protein having the amino acid sequence of SEQ ID NO: 42 or a sequence having at least 90%, 91%, 92%, 93%, 94%, 95%, 96%, 97%, 98% or at least 99% sequence identity thereto; and the (iii) one or more CRISPR-Cas portions is/are fused to portion (iv) as third fusion protein.

In another embodiment of the first aspect, the diversifying base editor comprises an amino acid molecule selected from any one of SEQ ID NO: 1-27, 52 or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

SEQ ID NO: 1 is a Cas9-based DBE comprising an hA3A, a dimeric ecTadA/ecTadA7.10, an enCas9 with an additional D10A nickase mutation, and three repeats of an SV40 NLS: hA3A 48aa-XTEN-linker-ecTadA-32aa-XTEN-linker-ecTadA7.10-32aa-XTEN-linker-enCas9 (D10A)-SV40 NLS(3x).

SEQ ID NO: 2 has the same architecture as SEQ ID NO: 1 but comprises an LbCas12a (D156R/D832A) instead of Cas9: hA3A-48aa-XTEN-linker-ecTadA-32aa-XTEN-linker-ecTadA7.10-32aa-XTEN-linker-LbCas12a(D156R/D832A)-SV40 NLS(3x).

SEQ ID NO: 3 has an additional E795L mutation: hA3A-48aa-XTEN-linker-ecTadA-32aa-XTEN-linker-ecTadA7.10-32aa-XTEN-linker-LbCas12a(D156R/E795L/D832A)-SV40 NLS(3x).

SEQ ID NO: 4 has the same architecture as SEQ ID NO: 2 but comprises an hA3A(R128A) cytosine deaminase mutant: hA3A(R128A)-48aa-XTEN-linker-ecTadA-32aa-XTEN-linker-ecTadA7.10-32aa-XTEN-linker-LbCas12a(D156R/D832A)-SV40 NLS(3x).

SEQ ID NO: 5 has the same architecture as SEQ ID NO: 2 but comprises a dimeric TadA8e(V106W) adenine deaminase mutant: hA3A-48aa-XTEN-linker-ecTadA-32aa-XTEN-linker-ecTadA8e(V106W)-32aa-XTEN-linker-LbCas12a(D156R/D832A)-SV40 NLS(3x).

SEQ ID NO: 6 comprises both the hA3A(R128A) and a dimeric TadA8e(V106W): hA3A(R128A)-48aa-XTEN-linker-ecTadA-32aa-XTEN-linker-ecTadA8e(V106W)-32aa-XTEN-linker-LbCas12a(D156R/D832A)-SV40 NLS(3x).

SEQ ID NO: 7 comprises an N-terminal and a C-terminal dpNLS and a monomeric TadA8e adenine deaminase: dpNLS-hA3A-48aa-XTEN-linker-TadA8e-32aa-XTEN-linker-LbCas12a(D156R/D832A)-dpNLS.

SEQ ID NO: 8 has the same architecture as SEQ ID NO: 7 but comprises an additional K932/N933 mutation in the LbCas 12a: dpNLS-hA3A-48aa-XTEN-linker-TadA8e-32aa-XTEN-linker-LbCas12a(D156R/D832A/K932G/N933G)-dpNLS.

SEQ ID NO: 9 has the same architecture as SEQ ID NO: 7 but comprises an additional E795L mutation in the LbCas12a: dpNLS-hA3A-48aa-XTEN-linker-TadA8e-32aa-XTEN-linker-LbCas12a(D156R/E795L/D832A)-dpNLS.

SEQ ID NO: 10 has the same architecture as SEQ ID NO: 7 but the linker region between portion (ii) and portion (iii) is a (GGGGS)₅ linker: dpNLS-hA3A-48aa-XTEN-linker-TadA8e-(GGGGS)₅-LbCas12a(D156R/D832A)-dpNLS.

SEQ ID NO: 11 has the same architecture as SEQ ID NO: 7 but comprises a monomeric TadA9 adenine deaminase: dpNLS-hA3A-48aa-XTEN-linker-TadA9-32aa-XTEN-linker-LbCas12a(D156R/D832A)-bdpNLS.

SEQ ID NO: 12 has the same architecture as SEQ ID NO: 11 but the linker region between portion (ii) and portion (iii) is a (GGGGS)₆ linker: dpNLS-hA3A-48aa-XTEN-linker-TadA9-(GGGGS)₆-LbCas12a(D156R/D832A)-dpNLS.

SEQ ID NO: 13 has the same architecture as SEQ ID NO: 12 but comprises a hA3A(W104A/P134Y) mutant: dpNLS-hA3A(W104A/P134Y)-48aa-XTEN-linker-TadA9-(GGGGS)₆-LbCas12a(D156R/D832A)-dpNLS.

SEQ ID NO: 14 has the same architecture as SEQ ID NO: 12 but comprises a hA3A(Y130F) mutant: dpNLS-hA3A(Y130F)-48aa-XTEN-linker-TadA9-(GGGGS)₆-LbCas12a(D156R/D832A)-dpNLS.

SEQ ID NO: 15 has the same architecture as SEQ ID NO: 10 but comprises a uracil glycosylase inhibitor portion and a (GGGGS)₆: dpNLS-hA3A-48aa-XTEN-linker-TadA8e-(GGGGS)₆-LbCas12a(D156R/D832A)-UGI-dpNLS.

SEQ ID NO: 16 has the same architecture as SEQ ID NO: 10 but the linker region between portion (ii) and portion (iii) is a (GGGGS)₆ linker and that it comprises an E. coli uracil-N-glycosylase portion: dpNLS-eUNG-hA3A-48aa-XTEN-linker-TadA8e-(GGGGS)₆-LbCas12a(D156R/D832A)-dpNLS.

SEQ ID NO: 17 has the same architecture as SEQ ID NO: 2 but comprises an LbCas12a(D832A/D156R/G532R/K538R) mutant (enCas 12a(D832)): hA3A-48aa-XTEN-linker-ecTadA-32aa-XTEN-linker-ecTadA7.10-32aa-XTEN-linker-enCas12a(D832)-SV40 NLS(3x).

SEQ ID NO: 18 has the same architecture as SEQ ID NO: 17 but comprises a dimeric TadA8e(V106W): hA3A-48aa-XTEN-linker-ecTadA-32aa-XTEN-linker-TadA8e(V106W)-32aa-XTEN-linker-enCas12a(D832)-SV40 NLS(3x).

SEQ ID NO: 19 has the same architecture as SEQ ID NO: 17 but comprises hA3A (R128A): hA3A (R128A)-48aa-XTEN-linker-ecTadA-32aa-XTEN-linker-ecTadA7.10-32aa-XTEN-linker-enCas12a(D832)-SV40 NLS(3x).

SEQ ID NO: 20 comprises both the hA3A(R128A) and dimeric TadA8e(V106W): hA3A(R128A)-48aa-XTEN-linker-ecTadA-32aa-XTEN-linker-TadA8e(V106W)-32aa-XTEN-linker-enCas12a(D832)-SV40 NLS(3x).

SEQ ID NO: 21 has the same architecture as SEQ ID NO: 17 but comprises an additional E795L mutation in the enLbCas 12a: hA3A(R128A): hA3A-48aa-XTEN-linker-ecTadA-32aa-XTEN-linker-ecTadA7.10-32aa-XTEN-linker-enCas12a(E795L/D832)-SV40 NLS(3x).

SEQ ID NO: 22 comprises a dpNLS, an, a monomeric TadA9, a (GGGGS)₅ linker region and a Rad51ssDBD: dpNLS-hA3A-48aa-XTEN-linker-TadA9-(GGGGS)₅-Rad51ssDBD-LbCas12a(D156R/D832A)-dpNLS.

SEQ ID NO: 23 comprises a dpNLS, a monomeric TadA9 and a (GGGGS)₅ linker region: dpNLS-hA3A-48aa-XTEN-linker-TadA9-(GGGGS)₅-LbCas12a(D156R/D832A)-dpNLS.

SEQ ID NO: 24 comprises a dpNLS, an hA3A(W104A/P134Y), a monomeric TadA9 and a (GGGGS)₅ linker region: dpNLS-hA3A (W104A/P134Y)-48aa-XTEN-linker-TadA9-(GGGGS)₅-LbCas12a(D156R/D832A)-dpNLS.

SEQ ID NO: 25 comprises a dpNLS, an hA3A(Y130F), a monomeric TadA9 and a (GGGGS)₅ linker region: dpNLS-hA3A (Y 130F)-48aa-XTEN-linker-TadA9-(GGGGS)₅-LbCas12a(D156R/D832A)-dpNLS.

SEQ ID NO: 26 has the same architecture as SEQ ID NO: 10 but comprises a uracil glycosylase inhibitor portion: dpNLS-hA3A-48aa-XTEN-linker-TadA8e-(GGGGS)₅-LbCas12a(D156R/D832A)-UGI-dpNLS.

SEQ ID NO: 27 has the same architecture as SEQ ID NO: 10 but comprises an E. coli uracil-N-glycosylase portion: dpNLS-eUNG-hA3A-48aa-XTEN-linker-TadA8e-(GGGGS)₅-LbCas12a(D156R/D832A)-dpNLS.

SEQ ID NO: 52 comprises a dpNLS, an hA3A, a monomeric TadA9 and a (GGGGS) 3 linker region: dpNLS-hA3A-48aa-XTEN-linker-monomeric TadA9-(GGGGS)₃-LbCas12a(D156R/D832A)-dpNLS (SEQ ID NO: 52).

In a second aspect, there may be provided an edited cell, tissue, organ, material or whole organism obtained by or obtainable by a method according to the first aspect.

In a third aspect, there may be provided a diversifying base editor, or a diversifying base editor complex additionally comprising at least one suitable guide RNA, or at least one nucleic acid molecule encoding the same, wherein the diversifying base editor is as defined in the first aspect.

As it is known to the skilled person, guide RNA scaffolds for different types of CRISPR nucleases exist and these can be individually designed to interact with a PAM motif at/near the target base to be edited/exchanged.

In a fourth aspect, there may be provided, a vector or expression construct, or more than one vectors and expression constructs, each vector and/or expression construct comprising the at least one nucleic acid molecule of the third aspect, wherein different portions of the diversifying base editor are encoded on the same vector or expression construct or on different vectors or expression constructs, and/or wherein the diversifying base editor, or portions thereof, and the at least one suitable guide RNA are encoded on the same vector or expression construct or on different vectors or expression constructs.

In a fifth aspect, there may be provided a cell comprising at least one diversifying base editor or at least one diversifying base editor complex, or at least one nucleic acid molecule encoding the same, of the third aspect; or at least one vector or expression construct of the fourth aspect; wherein the cell is a prokaryotic cell, including a bacterial cell or an archaea cell, or a eukaryotic cell, including a an insect cell, a mammalian cell or plant cell, including a plant protoplast, preferably wherein the cell is a plant cell, including a plant protoplast, optionally wherein the plant cell, including a plant protoplast, is a cell of, or originating from, a plant selected from wherein the at least one target cell is a plant cell of, or originating from, a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

Preferred plants are Abelmoschus spp., Allium spp., Apium graveolens, Asparagus officinalis, Avena spp. (e.g. Avena sativa, Avena fatua, Avena byzantina, Avena fatua var. sativa, Avena hybrida), Beta vulgaris, Brassica spp. (e.g. Brassica napus, Brassica rapa ssp. [canola, oilseed rape, turnip rape]), Capsicum spp., Citrullus lanatus, Cucumis spp., Cynara spp., Daucus carota, Glycine spp. (e.g. Glycine max, Soja hispida or Soja max), Gossypium hirsutum, Helianthus spp. (e.g. Helianthus annuus), Hordeum spp. (e.g. Hordeum vulgare), Lactuca sativa, Medicago sativa, Oryza spp. (e.g. Oryza sativa, Oryza latifolia), Pennisetum sp., Saccharum spp., Secale cereale, Solanum spp. (e.g. Solanum tuberosum, Solanum integrifolium or Solanum lycopersicum), Sorghum bicolor, Spinacia spp., Triticum spp. (e.g. Triticum aestivum, Triticum durum, Triticum turgidum, Triticum hybernum, Triticum macha, Triticum sativum, Triticum monococcum or Triticum vulgare), or Zea mays.

Preferred plants, in certain embodiments, may also be selected from Brassica spp. (e.g. Brassica napus, Brassica rapa ssp. [canola, oilseed rape, turnip rape]), Capsicum spp., Glycine spp. (e.g. Glycine max, Soja hispida or Soja max), Gossypium hirsutum, Helianthus spp. (e.g. Helianthus annuus), Oryza spp. (e.g. Oryza sativa, Oryza latifolia), Solanum spp. (e.g. Solanum tuberosum, Solanum integrifolium or Solanum lycopersicum), Triticum spp. (e.g. Triticum aestivum, Triticum durum, Triticum turgidum, Triticum hybernum, Triticum macha, Triticum sativum, Triticum monococcum or Triticum vulgare), or Zea mays.

In a sixth aspect, there may be provided a kit comprising at least one diversifying base editor or at least one diversifying base editor complex, or at least one nucleic acid molecule encoding the same, of the third aspect; or at least one vector or expression construct of the fourth aspect; or at least one cell of the fifth aspect, and optionally instructions for use and necessary buffers, equipment and reagents.

The diversifying base editor, diversifying base editor complex comprising guide RNA, the nucleic acid molecule encoding the same, and/or the vector or expression construct is provided in a functional form, e.g., including stabilizers, cofactors, means for introducing the same into a target cell or tissue and the like.

In a seventh aspect, there may be provided a use of at least one diversifying base editor or at least one diversifying base editor complex, or at least one nucleic acid molecule encoding the same, of the third aspect; or at least one vector or expression construct of the fourth aspect; or at least one cell of the fifth aspect; or of at least one kit of the sixth aspect; for targeted directed evolution of at least one target nucleic acid segment, preferably in planta targeted directed evolution of at least one target nucleic acid segment, including a use for optimizing or modifying a trait in a plant, including the optimization or modification of a yield-related trait, or a disease or pathogen resistance related trait, wherein the disease is caused by, or the pathogen is selected from a virus, a bacterium, a fungus, a nematode, or an insect, or a herbicide-resistance related trait, or an abiotic-stress related trait, including a salinity or drought stress related trait, further including further including a use for identification of at least one gene and/or genomic locus being associated with at least one trait of interest.

Targeted directed evolution refers to any strategy of diversification of a target nucleic acid segment followed by genotypic and/or phenotypic screening and/or selection, optionally comprising the application of selective pressure, typically performed as iterative rounds of mutagenesis, wherein each round of mutagenesis may comprise the steps of regenerating an organism, including a plant, from the cell, tissue and/or material, including a plant protoplast or callus, used for mutagenesis, and/or regenerating plant material, e.g. via callus culture, or by direct rooting/shooting, and/or for crossing, including backcrossing.

In an eighth aspect, there is provided a Brassica Napus acetolactate synthase (ALS) 3 protein comprising a D358N and a R359H mutation or an Arabidopsis thaliana acetohydroxyacid synthase (AHAS) protein comprising a D376N and a R377H mutation.

In one embodiment, the Brassica Napus ALS3 protein comprises or consists of an amino acid sequence of SEQ ID NO: 77 or a sequence having at least 90%, 91%, 92%, 93%, 94%, 95%, 96%, 97%, 98% or at least 99% sequence identity thereto.

In a ninth aspect, there is provided a nucleic acid molecule encoding the ALS3 or AHAS protein of the eighth aspect.

In one embodiment, the nucleic acid molecule comprises or consists of the sequence of SEQ ID NO: 76 or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In a tenth aspect, there is provided a plant or plant cell comprising and/or encoding an ALS3 protein or AHAS protein of the eighth aspect or a nucleic acid molecule of the ninth aspect.

In one embodiment, the plant or plant cell is a Brassica Napus or Arabidopsis thaliana plant or plant cell.

### EXAMPLES

### Example 1: Cloning Methods and Plasmid Construction

Unless indicated otherwise, cloning procedures carried out for the purpose of the current invention including restriction digest, agarose gel electrophoresis, purification and ligation of nucleic acids, transformation, selection and cultivation of bacterial cells were performed as described in the literature available to the skilled person since long (cf., Sambrook, Fritsch and Maniatis, 1989). Sequence analysis of recombinant DNA was performed by LGC Genomics (Berlin, Germany) using the Sanger technology. Restriction endonucleases and Gibson Assembly reagents used to construct the various expression vectors are from New England Biolabs (Ipswich, MA, USA). Oligonucleotides are synthesized by Integrated DNA Technologies (Coralville, IA, USA). Codon-optimized genes are from Genewiz (South Plainfield, NJ, USA).

All base editors were optimized for expression in plant cells and the codon usage of wheat high-expressing genes.

All expression vectors include the maize polyubiquitin (Ubi) promoter (Seq ID NO: 28) for constitutive expression, located upstream of the coding sequence, and a fragment of the 3′ untranslated region of the octopine-type Ti plasmid gene 7 of Agrobacterium tumefaciens (Seq ID NO: 29) or the 35S gene of Cauliflower mosaic virus (Seq ID NO: 30) at the 3′end. gRNA expression cassettes containing a Cas12a guide RNA composed of a truncated glycine-tRNA (Seq ID NO: 31), a 21-bp direct repeat sequence (Seq ID NO: 32), a 23-bp protospacer site targeting the rice OsAAT gene (LOC_Os01g55540.1) (Seq ID NO: 33), and the rice polymerase III terminator sequence (comprising eight “Ts” in a row) were ordered as synthetic fragments and cloned into a standard E. coli vector (pUC derivative) via EcoRV blunt end ligation. Expression of the gRNA is driven by the polymerase III-type promoter of the rice U6 snRNA gene (Seq ID NO: 35).

All plasmids were transformed in E. coli for propagation and isolated using a ZymoPure II Plasmid Gigaprep kit for DNA purification (Zymo Research, Irvine, CA, USA).

### Example 2: Design of a Cas9 Diversifying Base Editor

To design a Cas9-based diversifying base editor (Cas9 DBE), an existing Cas9 dual base editor, STEME-1 (Li et al., 2020), was optimized to allow greater sequence diversification. To produce the construct DBE-1, the Cas9 (D10A) nickase in STEME-1 was exchanged with enCas9, a variant of Cas9 (D10A) with enhanced DNA dissociation (Slaymaker et al., 2016), the UGI domain was removed and the nucleoplasmin and single SV40 NLS were replaced with three C-terminal repeats of SV40 NLS (see FIG. 1A). Both STEME-1 and the DBE-1 were optimized for expression in monocots and their respective activities were determined in rice protoplasts by measuring the total number of base edits at the AAT target site.

Transformation of rice protoplast cells was performed as described by Shan et al., 2014 with minor modifications. Protoplasts were isolated from the sheaths of 3-week-old aseptically grown rice seedlings. Healthy stems and sheaths were bundled in stacks of 20 and cut into fine strips with a sharp razor blade. The strips were then infiltrated with cell wall-dissolving enzyme solution (1.5% cellulase R10 and 0.75% macerozyme R10 in 10 mM KCl and 0.6 M mannitol, pH 7.5) and incubated overnight in the dark with gentle shaking (40 rpm) at 24° C. After enzymatic digestion, the released protoplasts were collected by filtering the mixture through 40-μm nylon meshes and resuspended in W5 solution. The resuspended protoplasts were washed with W5 solution, after which the cell pellet was suspended in MMG solution at a density of 2.5 million cells/ml. For transformation, 200 μl of cells (5×105) were mixed with 20 μg plasmid DNA and 220 μl of freshly prepared polyethylene glycol (PEG) solution. The mixture was incubated for 15-20 min in the dark. After removing the PEG solution, the protoplasts were resuspended in 2 ml of WI solution, transferred into six-well plates, and incubated at 24° C. for at least 48 h.

Both STEME-1 and DBE-1 were co-transfected with an AAT-targeting Cas9 guide RNA construct comprising from 5′ to 3′ end: a truncated tRNA, a first mature direct repeat sequence, the spacer RNA, a second mature direct repeat sequence, and a poly-T tail (T-stretch terminator). Three days post transfection, protoplasts were harvested by centrifugation and genomic DNA was extracted using either Phire Tissue Direct PCR extraction buffer (Thermo Fisher Scientific) or the Qiagen DNeasy Plant kit. The AAT target region was amplified by PCR using primers SEQ ID NO: 36 and SEQ ID NO: 37 and subjected to amplicon deep sequencing.

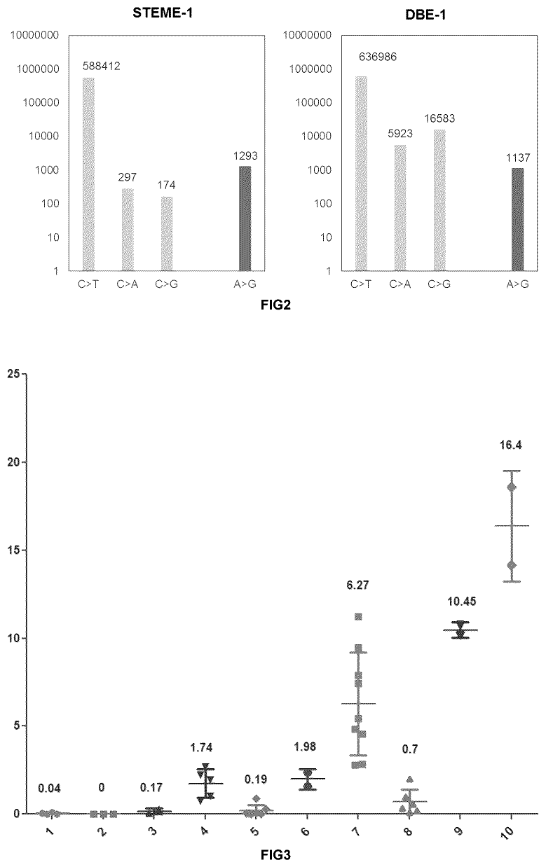

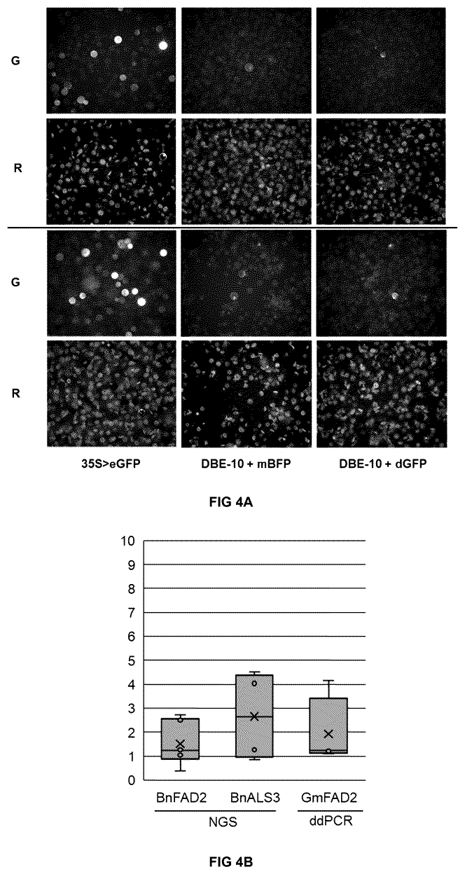

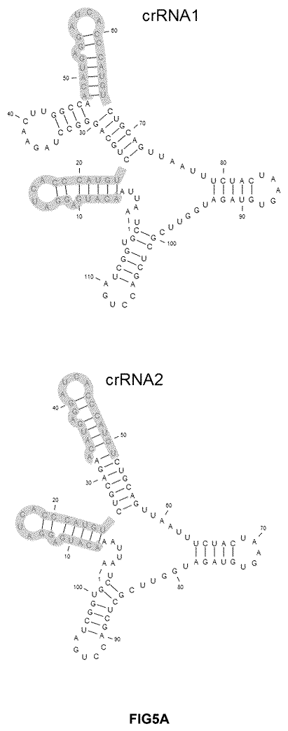

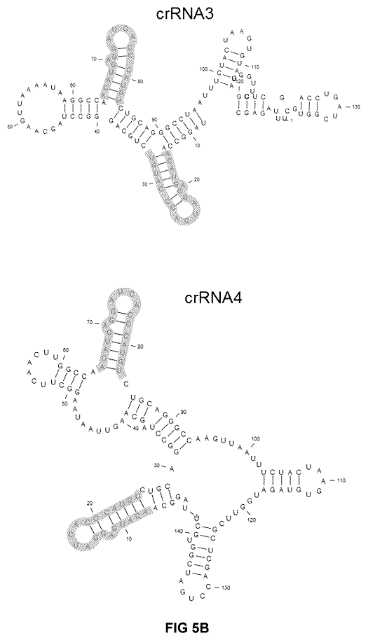

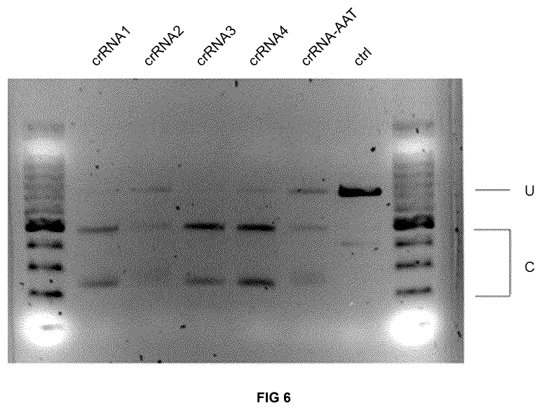

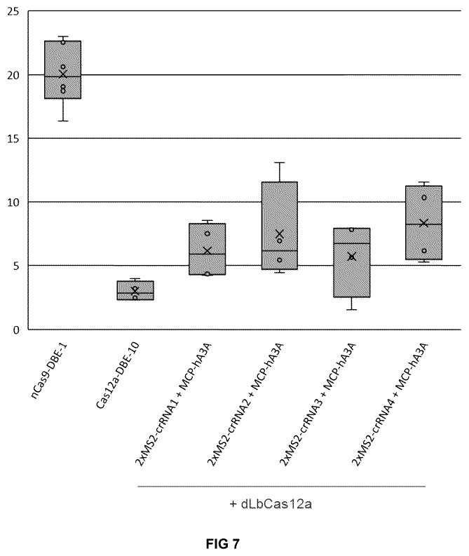

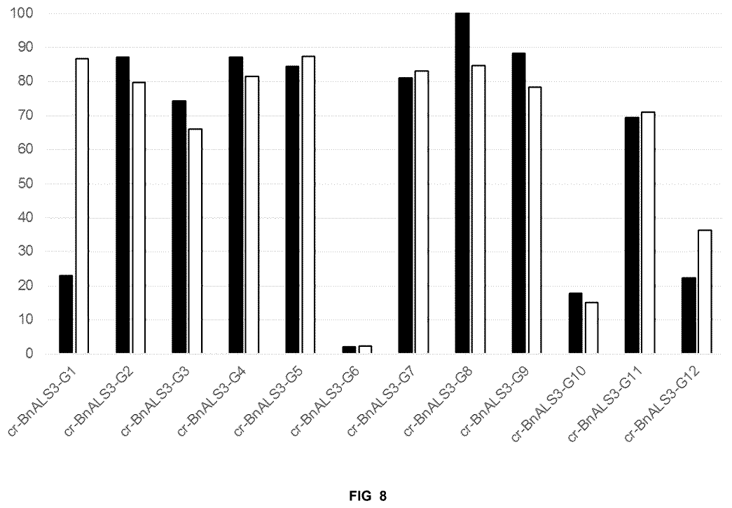

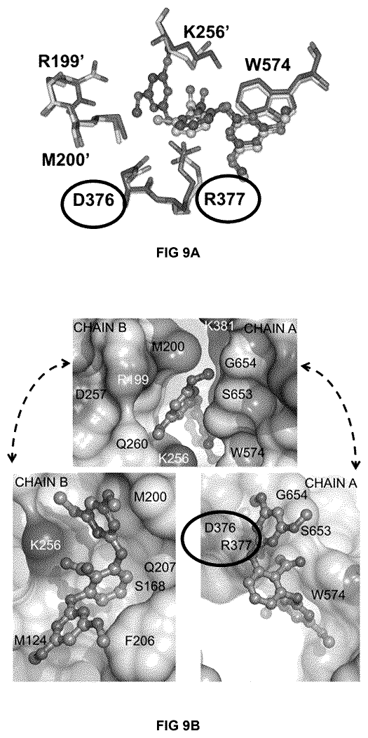

While the total base editing efficiency of STEME-1 and DBE-1 was very similar (49.43% versus 50.19%), DBE-1 showed an overall broader mutation spectrum with strongly increased C to A and C to G substitutions (see FIG. 1B and FIG. 2). Moreover, DBE-1 exhibited a slightly enlarged C-to-T base-editing window spanning position C1 to C16 as opposed to C7-C16 for STEME-1 (counting the end distal to the PAM as position 1, data not shown). Consistent with the broader mutation spectrum and enlarged editing window of DBE-1, we also found a higher number of identified alleles around the AAT target site as compared to STEME-1 transfected cells. Together, these results indicate that DBE-1 can increase the mutation diversity at a target site using a single guide RNA.

### Example 3: Development and Optimization of Cas12a Diversifying Base Editors

Based on the Cas9 DBE-1 architecture, a series of expression vectors encoding different Cas12a diversifying base editors (Cas12a DBEs) were constructed. Each of these expression constructs contained different modifications with respect to the NLS configuration, the adenosine deaminase portion(s), the cytosine deaminase portion; the Cas 12a portion and/or the protein linker connecting the adenosine portion to Cas 12a (see FIG. 3).

All Cas12a DBEs were optimized for expression in monocot plants and transcribed from a constitutive maize Ubi promoter. To examine their base-editing activities, each of the Cas12a DBE constructs was transfected in rice protoplasts along with a guide RNA expression construct including a truncated glycine-tRNA and two mature direct repeats 5′ and 3′ of the spacer. Some base editor constructs were tested in combination with the LbCas 12a R1138A mutation, which is expected to perturb base editing either via nicking of the non-target strand or through residual DSB nuclease activity (Yamano et al., 2016). Total base editing efficiencies of selected Cas12A DBE architectures as measured by amplicon deep sequencing are shown in FIG. 3 and Table 1.

TABLE 1

| | Cas12a DBE | Relative DBE |
|---|---|---|
| Treatment | construct | activity (%) |
| Cas12a DBE - TadA8e | Cas12a-DBE-7 | 79.73 ± 9.45 |
| Cas12a DBE - TadA8e - (GGGGS)5x | Cas12a-DBE-10 | 100 ± 13.48 |
| Cas12a DBE - TadA9 - (GGGGS)5x | Cas12a-DBE-11 | 115.04 ± 12.31 |
| Cas12a DBE - TadA9 - (GGGGS)5x - | Cas12a-DBE-12 | 25.72 ± 0.66 |
| hA3A(Y130F) | | |
| Cas12a DBE - TadA9 - (GGGGS)3x | Cas12a-DBE-13 | 75.56 ± 16.08 |
| Cas12a DBE - TadA8e - (GGGGS)5x + | Cas12a-DBE-10 | 134.13 ± 1.67 |
| 35S > eUNG | | |
| eUNG-Cas12a DBE - TadA8e - (GGGGS)5x | Cas12a-DBE-14 | 0.87 ± 0.04 |
| 35S > eUNG | nd | 0.78 ± 0.08 |

Table 1 shows the results of different LbCas12a DBE constructs at the OsAAT target site in rice protoplasts. The editing efficiency of the different constructs is expressed relative to that shown by LbCas12a-DBE-10 (see FIG. 3, SEQ ID NO: 10). The different used LbCas12a-DBE architectures are:

DBE-7: dpNLS (dual portion nuclear localization signal)-hA3A-48aa-XTEN-linker-monomeric TadA8e-32aa-XTEN-linker-LbCas12a(D156R/D832A)-dpNLS (SEQ ID NO: 7)

DBE-10: dpNLS-hA3A-48aa-XTEN-linker-monomeric TadA8e-GGGGS-linker-(5x)-LbCas12a(D156R/D832A)-dpNLS (SEQ ID NO: 10)

DBE-11: dpNLS-hA3A-48aa-XTEN-linker-monomeric TadA9-GGGGS-linker-(5x)-LbCas12a(D156R/D832A)-dpNLS (SEQ ID NO: 23)

DBE-12: dpNLS-hA3A (Y130F)-48aa-XTEN-linker-monomeric TadA9-GGGGS-linker-(5x)-LbCas12a(D156R/D832A)-dpNLS (SEQ ID NO: 25)

DBE-13: dpNLS-hA3A-48aa-XTEN-linker-monomeric TadA9-GGGGS-linker-(3x)-LbCas12a(D156R/D832A)-dpNLS (SEQ ID NO: 52)

DBE-14: dpNLS-eUNG-hA3A-48aa-XTEN-linker-monomeric TadA8e-GGGGS-linker-(5x)-LbCas12a(D156R/D832A)-dpNLS (SEQ ID NO: 27)

Several rounds of optimization demonstrated that the following modifications can increase base diversification:
use of monomeric TadA adenine deaminase instead of dimeric TadA adenine deaminase
use of monomeric TadA9 deaminase instead of TadA8e deaminase
use of (GGGGS)₅ as linker between portion (ii) and portion (iii)
use of the bipartite NLS SEQ ID NO:49 instead of three repeats of SV40 NLS
use of a dpNLS at both the N-terminus and the C-terminus

The highest level of base editing was determined for construct 11 (SEQ ID NO: 23; see Table 1) comprising bipartite SV40 NLS (SEQ ID NO: 49) at both 5′ and 3′ ends, a hA3A cytosine deaminase domain, monomeric TadA9 as an adenosine deaminase domain and a (GGGGS)₅ linker connecting TadA9 to catalytically inactive LbCas12a harboring the D156R mutation.

The second highest level of base editing (averaging 16.4%) was determined for construct 10 (SEQ ID NO: 10; see FIG. 3) comprising a bipartite NLS (SEQ ID NO: 49) at both 5′ and 3′ ends, a hA3A cytosine deaminase domain, a monomeric TadA8e as an adenosine deaminase domain and a (GGGGS)₅ linker connecting TadA8e to catalytically inactive LbCas 12a harboring the D156R mutation.

Interestingly, introducing the K932G/N933G mutations in the Cas12a domain, which were previously hypothesized to enhance base editing by nicking the target strand (Paul et al., 2021), reduced efficiency of base substitutions by strongly increasing indel formation (construct 8; see FIG. 3). Also, substituting glutamic acid at position 795 in Cas 12a by a leucine (E795L), an amino acid change found to enhance Cas12a activity in mammalian cells (WO2020/172502 A1), failed to substantially increase base substitution rates in rice protoplasts (constructs 3, 6 and 9; see FIG. 3), while the introduction of a Y130F mutation in the hA3A domain or the use of a tri-GGGGS linker between the adenosine deaminase domain and Cas12a lowered editing rates (see Table 1). Interestingly, the efficiency of DBE-10 could be further enhanced via co-delivery with an Escherichia coli-derived uracil DNA N-glycosylase (eUNG) expressed in trans from a strong 35S promoter (see Table 1), suggesting that the creation of abasic sites and subsequent induction of base excision DNA repair promotes target diversification by DBEs. Yet, in contrast to findings for Cas9 (Kurt et al., 2021), adding eUNG to the N-terminus of Cas12a DBE-10 had a strong negative impact on editing activity (see Table 1).

Further modifications are currently being tested, including the effect of fusing C-terminal UGI and UNG domains to Cas12a DBE, the impact on mutagenesis efficiency of a (GGGGS)₆ linker between portion (ii) and portion (iii), the introduction of W104A/P134Y mutations in the hA3A domain and the impact on base substitution rates of a non-sequence specific ssDNA-binding domain between portion (ii) and portion (iii).

### Example 4: LbCas12a-DBE Activity in Soybean and Oilseed Rape

To determine the activity of LbCas 12a-DBE in dicot plants, additional experiments using oilseed rape (Brassica napus) and soybean (Glycine max) protoplasts were performed. Oilseed rape protoplasts were isolated from the leaves of 4-to 7-week-old aseptically grown plants. Healthy leaves were cut into fine strips with a sharp razor blade. The strips were infiltrated with cell wall-dissolving enzyme solution (0.25% cellulase R10 and 0.25% macerozyme R10) and incubated overnight in the dark with gentle shaking (40 rpm) at 24° C. After enzymatic digestion, the released protoplasts were collected by filtering the mixture through 40-μm nylon meshes and resuspended in W5 solution. The resuspended protoplasts were kept on ice and allowed to settle by gravity, after which the cell pellet was resuspended in MMG. For transformation, 200 μl of cells (2.5×105) were mixed with 20 μg plasmid DNA and 220 μl of freshly prepared polyethylene glycol (PEG) solution. The mixture was incubated for 15-20 min in the dark. After removing the PEG solution, the protoplasts were resuspended in 2 ml of W5 solution and incubated at 24° C. Soybean protoplasts were isolated from the unifoliate leaves of 6-day-old seedlings and transfected essentially as described for oilseed rape. After removing the PEG solution, the protoplasts were resuspended in 2 ml of WI solution.

Cas12a-DBE activity was first evaluated using two different reporter systems. The first reporter is activated after C-to-T editing for conversion of blue fluorescent protein (BFP) to green fluorescent protein (GFP) conversion, which requires changing codon 66 from CAC (histidine) to TAC (tyrosine; cf. SEQ ID NO: 53)). The second assay detects A-to-G editing of an inactivated GFP reporter harboring an early stop codon resulting from changing codon 110 from CGA (arginine) to TAG (cf. SEQ ID NO: 54). Editing of the BFP or inactivated GFP reporter will restore the GFP coding sequence and result in GFP fluorescence.

Oilseed rape protoplasts were co-transfected with 3 vectors: (1) a vector encoding either BFP (SEQ ID NO: 53) or inactivated GFP (SEQ ID NO: 54), both of which contain an engineered TTTC Cas12a PAM site (due to a T62S substitution in BFP and a silent AAG to AAA mutation at K114 in GFP) (2) a Cas12a-DBE expression construct comprising hA3A as a cytosine deaminase domain and TadA9 as an adenosine deaminase domain and a penta-GGGGS linker connecting TadA8e to a dLbCas12a (D156R) module located 3′ of TadA8e (i.e. DBE-10; SEQ ID NO: 10) and (3) a vector encoding a Cas 12a gRNA targeting either the BFP or GFP reporter and containing two mature direct repeats 5′ and 3′ of the spacer (SEQ ID NO: 55; SEQ ID NO: 56). The DBE-10 vector included the Arabidopsis ubiquitin10 promoter for constitutive expression (SEQ ID NO: 57), while expression of the gRNA was driven by the polymerase III-type promoter of the Arabidopsis U6 snRNA gene (SEQ ID NO: 58). As a positive control, protoplasts were transfected with a construct expressing wild-type eGFP under control of a strong cauliflower mosaic virus (CaMV) 35S promoter (SEQ ID NO: 59). As a negative control the Cas12a-DBE-10 fusion protein was tested without the gRNA. Fluorescence imaging at 2 days post transfection revealed approximately 35% GFP-fluorescent cells in the positive control and 3.5% and 2.1% with dCas12a-DBE-10 and the BFP and dGFP reporters, respectively (see FIG. 4A). Importantly, no GFP-positive cells could be observed in the absence of the gRNA (data not shown).

To confirm Cas12a-DBE activity at endogenous sites, the pAtUbi10>DBE-10 expression construct was co-transfected into oilseed rape or soybean protoplasts along with a Cas12a gRNA targeting the BnFAD2 (gRNA: SEQ ID NO: 60), BnALS3 (gRNA: SEQ ID NO: 61) or GmFAD2 (gRNA: SEQ ID NO: 62) genes. Transfected oilseed rape protoplasts were cultured in alginate and editing efficiencies were determined at 14 days post transfection by deep amplicon sequencing. Conversely, soybean protoplasts were incubated in WI solution for 72 hours and analyzed via droplet digital PCR. As shown in FIG. 4B, transfection of DBE-10 resulted in successful editing of all 3 genes tested, with up to 4.5% of the NGS or ddPCR reads showing C-to-T and/or A-to-G base changes (average of 1.51%, 2.66% and 1.92% for BnFAD2, BnALS3 and GmFAD2, respectively). Together with the data in rice protoplasts, these results show that Cas12a-DBE is active in both monocot and dicot plants.

### Example 5: MS2 Tagging for Diversifying Base Editors

In order to develop MS2 tagging strategies for Cas12a-DBEs four different Cas12a guide RNAs harboring two MS2 stem-loops at the 5′ end of the guide were designed (see FIG. 5A and FIG. 5B; SEQ ID NO: 38, SEQ ID NO: 39, SEQ ID NO: 40, SEQ ID NO: 41). To test the effect of the additional MS2 stem-loops on the activity of the Cas12a-crRNA complex, an in vitro digest with purified OsAAT PCR product targeted by the different guide RNA designs was performed. The sequence of the gRNA target site is listed as SEQ ID NO: 33

25 μl reactions were prepared by mixing 500 ng of purified OsAAT PCR substrate, 2 μl pre-assembled Cas12a RNP including 29 picomoles of crRNA and 22 picomoles of protein, and 2.5 μl 10×NEB buffer 2.1. Reactions were incubated for 60 minutes at 37° C., heat inactivated at 85° C. for 2 minutes, and separated on a 1% agarose gel containing 1/100 (v/v) SYBR-Safe (Invitrogen). A shift in the position of the OsAAT PCR product indicates successful cleavage. As shown in FIG. 6, all four MS2-modified guide RNA designs yielded bands indicative of substrate cleavage similar to those seen in the positive control sample (i.e. non-modified guide RNA). Comparable levels of indel formation were also found in rice protoplasts co-transfected with LbCas12a and either untagged gRNA or one of the four MS2-modified variants (see Table 2).

TABLE 2

| Nuclease | OsAAT-targeting gRNA | Indel efficiency (%) |
|---|---|---|
| LbCas12a | None | 0.51 |
| LbCas12a | Untagged crRNA | 6.89 |
| LbCas12a | 2xMS2_crRNA_design1 | 4.92 |
| LbCas12a | 2xMS2_crRNA_design2 | 12.78 |
| LbCas12a | 2xMS2_crRNA_design3 | 11.36 |
| LbCas12a | 2xMS2_crRNA_design4 | 5.14 |

Table 2 shows the indel frequencies in rice protoplasts for an OsAAT target site induced by the four different Cas12a-MS2 guide RNAs shown in FIG. 5 compared to those induced by an untagged crRNA control.

Having confirmed that the addition of MS2 stem-loops does not affect the cleavage activity of Cas12a gRNAs, we next evaluated the impact of MS2 tagging on the level of base editing. To this end, rice protoplasts were co-transfected with an expression construct containing catalytically dead LbCas12a (D832A; SEQ ID NO: 63) together with one of the four gRNA bearing two MS2 hairpin-binding sites and a third vector encoding a fusion of the bacteriophage MS2 N55K coat protein (MCP) and the hA3A cytosine deaminase domain (SEQ ID NO: 43). The MCP-encoding sequence contained a N55K mutation that increases protein affinity to MS2 stem loops (Peabody, 1993). The base-editing activity of the different dCas12a-directed MS2-hA3A fusions was determined at three days post transfection by amplicon deep sequencing and compared to that of Cas 12a-DBE-10. While different MS2-gRNA designs exhibited varying mutation efficiencies depending on the target gene, recruitment of hA3A through dCas12a generally improved editing activity relative to that of the DBE-10 fusion protein (see FIG. 7 and Table 3). The biggest increase in editing was observed for the OsDEP1 target where the MS2-gRNA designs 3 and 4 (see FIG. 5) resulted in a respectively 8.25-fold and 7.42-fold average increase in editing efficiency compared to DBE-10. Together these results demonstrate that targeted recruitment of deaminases via MS2-modified gRNAs and catalytically inactive Cas12a can be exploited to boost the level of targeted random mutagenesis in plants.

TABLE 3

| | | Mutation efficiency |
|---|---|---|
| | OsDEP1- | (% of NGS reads |
| Cas12a module | targeting gRNA module | with base changes) |
| Cas12a-DBE-10 | Untagged crRNA | 0.68 ± 0.62 |
| dLbCas12a | 2xMS2_crRNA_design1 + | 4.48 ± 1.59 |
| | MCP-hA3A | |
| dLbCas12a | 2xMS2_crRNA_design2 + | 2.78 ± 0.15 |
| | MCP-hA3A | |
| dLbCas12a | 2xMS2_crRNA_design3 + | 5.61 ± 0.01 |
| | MCP-hA3A | |
| dLbCas12a | 2xMS2_crRNA_design4 + | 5.05 ± 033 |
| | MCP-hA3A | |
| | | Mutation efficiency |
| | OsACC- | (% of NGS reads |
| Cas12a module | targeting gRNA module | with base changes) |
| Cas12a-DBE-10 | Untagged crRNA | 0.27 ± 0.11 |
| dLbCas12a | 2xMS2_crRNA_design1 + | 0.78 ± 0.44 |
| | MCP-hA3A | |
| dLbCas12a | 2xMS2_crRNA_design2 + | 1.03 ± 0.19 |
| | MCP-hA3A | |
| dLbCas12a | 2xMS2_crRNA_design3 + | 1.03 ± 0.14 |
| | MCP-hA3A | |
| dLbCas12a | 2xMS2_crRNA_design4 + | 1.15 ± 0.31 |
| | MCP-hA3A | |

Table 3 shows the total base editing efficiency in rice protoplasts at the OsDEP1 and OsACC target sites of dLbCas12a-directed MS2-hA3A fusions with the four different Cas12a-MS2 guide RNA architectures shown in FIG. 5. Cas12a-DBE-10 refers to construct 10 in FIG. 3 (SEQ ID NO: 10). Mutation efficiency is expressed as the percentage of NGS reads with base changes.

### Example 6: Use of Cas12a-DBE for Directed Evolution of Novel Herbicide Tolerance in Oilseed Rape

Diversifying base editors hold great promise for rapidly improving agronomic traits via protein-directed evolution. To test the potential of Cas12a-DBEs for evolving novel herbicide tolerance, DBE-10 (SEQ ID NO: 10) was used for directed evolution of acetohydroxyacid synthase (AHAS, EC 2.2.1.6) in oilseed rape plants. AHAS, also referred to as acetolactate synthase (ALS), is the first enzyme in the pathway for biosynthesis of the branched-chain, essential amino acids valine, leucine and isoleucine. AHAS inhibitor herbicides have been widely used since their first introduction in the early 1980s owing to their broad-spectrum weed control at very low rates, low mammalian toxicity and wide crop selectivity. Twelve Cas12a gRNAs targeting the ALS3 gene of oilseed rape (Brassica napus) were designed, including 6 gRNAs with TTTV-3′ PAM sites and 6 gRNAs with TYTC-3′ PAMs (see Table 4). To test the activity of the designed gRNAs, individual guides together with LbCas12a (for targeting of TTTV PAMs) or LbCas12a-G532R/K595R (for targeting of TYTC PAMs) were transfected into oilseed rape protoplasts. Since amplicon deep sequencing showed high indel frequencies for most target sites (see FIG. 8)), a proof-of-concept experiment was initiated in which oilseed rape protoplasts were transformed with multiple ALS3-targeting gRNAs together with LbCas12a-DBE-10 or LbCas12a(G532R/K595R)-DBE-10. Transfected protoplasts were embedded in 1% alginate layers and cultured for at least two weeks at 24° C. before being plated on modified MS medium containing selective concentrations of the AHAS inhibitor bispyribac sodium salt. Approximately 3-4 weeks after plating, developing structures were transferred to MS regeneration medium and individual shoots were sequenced to retrieve the resistance-conferring mutations. Screening of protoplast-derived shoots transformed with a pooled gRNA library together with DBE-10 identified one resistant oilseed rape line that survived 1 nM bispyribac treatment (see Table 5). Sanger sequencing of this line revealed two mono-allelic D358N and R359H mutations, the former resulting from a single C-to-T transition and the latter caused by both C-to-T and A-to-G conversions indicative of simultaneous deaminase activities from DBE-10 (BnALS3_D358N/R359H coding sequence: SEQ ID NO: 76; BnALS3_D376N/R377H amino acid sequence: SEQ ID NO: 77). While the D358N mutation (corresponding to D376N in Arabidopsis thaliana AHAS, i.e. AtAHAS) is a known artificially generated resistance-endowing amino acid substitution, R359H (corresponding to R377H in AtAHAS) has been previously documented in resistant weeds (Yu and Powles, 2014). Both amino acid substitutions are predicted to result in protein structural changes that reduce the binding affinity of AHAS to bispyribac. As shown in FIG. 9, bispyribac possesses three aromatic rings which were found to adopt a twisted “S”-shaped conformation when bound to AtAHAS with the pyrimidinyl group inserted deepest into the herbicide binding site (Garcia et al., 2017). While one of the bispyribac methoxy groups forms contact with D376 of AtAHAS, the carboxylate group of bispyribac forms salt bridges to the side chains of R377. Together these results illustrate the ability of our DBEs to evolve novel herbicide-resistant alleles under selective pressure.

TABLE 4

| | Protospacer | | | |
|---|---|---|---|---|
| crRNA | Sequence | SEQ ID NO: | Nuclease | PAM |
| cr-BnALS3-G1 | GTTGATGTTCCTAAGGATATTCAG - | 64 | LbCas12a | TTTG |
| cr-BnALS3-G2 | GTGTTAGGTTTGATGACCGTGTCA | 65 | LbCas12a | TTTG |
| cr-BnALS3-G3 | CCGTGACACGGTCATCAAACCTAA | 66 | LbCas12a | TTTC |
| cr-BnALS3-G4 | TAGAACCGATCTTCCCATTGCATG | 67 | LbCas12a | TTTG |
| cr-BnALS3-G5 | AAAGTGCCACCACTTGGGATCATC | 68 | LbCas12a | TTTG |
| cr-BnALS3-G6 | GCGTCTCTTGGAAGGCGTCAGTAC | 69 | LbCas12a | ATTG |
| cr-BnALS3-G7 | ATTGCATGACCATCCCAAGATGCT | 70 | LbCas 12a- | TCCC |
| | | | RR | |
| cr-BnALS3-G8 | TGCAGATGCTTGGCATGCACGGGA | 71 | LbCas 12a- | TCCC |
| | | | RR | |
| cr-BnALS3-G9 | GGAGGTGCCTCCATGGAGATCCAC | 72 | LbCas 12a- | TCCC |
| | | | RR | |
| cr-BnALS3-G10 | \|TAACGTCCTCCCCCGTCACGAACA | 73 | LbCas 12a- | TCCG |
| | | | RR | |
| cr-BnALS3-G11 | TCGCCGGATGATCGGTACTGACGC | 74 | LbCas 12a- | TCCC |
| | | | RR | |
| cr-BnALS3-G12 | TCTCGTCGCCATCACAGGACAGGT | 75 | LbCas 12a- | TTCC |
| | | | RR | |

Table 4 shows an overview of the different Cas12a gRNAs used for targeted evolution of the BnALS3 gene of oilseed rape (Brassica napus).

TABLE 5

| | ALS inhibitor | SNP in | | Corresponding |
|---|---|---|---|---|
| Base editor | concentration | ALS3 allele | AA mutations | mutations in At |
| Cas12a-DBE-10 | 1 nM | Guide3: | mono-allelic | D376N |
| | bispyribac | GAC > AAC; | D358N + R359H | R377H |
| | | CGT > CAC | | |

Table 5 show the results of a Cas12a DBE-mediated directed evolution experiment in oilseed rape (Brassica napus) aimed at developing resistance against the AHAS-inhibiting herbicide bispyribac. Screening of protoplasts transformed with a pooled gRNA library (G1 to G6 of Table 4) and Cas12a DBE-10 (SEQ ID NO: 10) identified a herbicide-resistant line carrying two amino acid substitutions in the BnALS3 gene.

### REFERENCES

Eid A, Alshareef S, Mahfouz MM. CRISPR base editors: genome editing without double-stranded breaks. Biochem J. 2018 Jun 11;475(11):1955-1964. doi: 10.1042/BCJ20170793.
Fan J, Ding Y, Ren C, Song Z, Yuan J, Chen Q, Du C, Li C, Wang X, Shu W. Cytosine and adenine deaminase base-editors induce broad and nonspecific changes in gene expression and splicing. Commun Biol. 2021 Jul 16;4(1):882. doi: 10.1038/s42003-021-02406-5.
Garcia, MD et al. Comprehensive understanding of acetohydroxyacid synthase inhibition by different herbicide families. Proceedings of the National Academy of Sciences of the United States of America vol. 114,7 (2017): E1091-E1100. doi: 10.1073/pnas. 1616142114.
Gaudelli NM, Komor AC, Rees HA, Packer MS, Badran AH, Bryson DI, Liu DR. Programmable base editing of A⋅T to G⋅C in genomic DNA without DNA cleavage. Nature. 2017 Nov 23;551(7681):464-471. doi: 10.1038/nature24644.
Gautier A, Juillerat A, Heinis C, Correa IR Jr, Kindermann M, Beaufils F, Johnsson K. An engineered protein tag for multiprotein labeling in living cells. Chem Biol. 2008 Feb; 15(2):128-36. doi: 10.1016/j.chembiol.2008.01.007.
Hussain AF, Amoury M, Barth S. SNAP-tag technology: a powerful tool for site specific conjugation of therapeutic and imaging agents. Curr Pharm Des. 2013; 19(30):5437-42. doi: 10.2174/1381612811319300014.
Inobe T, Nukina N. Rapamycin-induced oligomer formation system of FRB-FKBP fusion proteins. J Biosci Bioeng. 2016 Jul; 122(1):40-6. doi: 10.1016/j.jbiosc.2015.12.004.
Jeong YK, Song B, Bae S. Current Status and Challenges of DNA Base Editing Tools. Mol Ther. 2020 Sep 2;28(9):1938-1952. doi: 10.1016/j.ymthe.2020.07.021.
Komor, A., Kim, Y., Packer, M. et al. Programmable editing of a target base in genomic DNA without double-stranded DNA cleavage. Nature 533, 420-424 (2016). https://doi.org/10.1038/nature17946.
Komor AC, Zhao KT, Packer MS, Gaudelli NM, Waterbury AL, Koblan LW, Kim YB, Badran AH, Liu DR. Improved base excision repair inhibition and bacteriophage Mu Gam protein yields C:G-to-T: A base editors with higher efficiency and product purity. Sci Adv. 2017 Aug 30;3(8): eaao4774. doi: 10.1126/sciadv.aao4774.
Kurt, IC et al. “CRISPR C-to-G base editors for inducing targeted DNA transversions in human cells.” Nature biotechnology vol. 39, 1 (2021): 41-46. doi: 10.1038/s41587-020-0609-x.
Lange A, McLane LM, Mills RE, Devine SE, Corbett AH. Expanding the definition of the classical bipartite nuclear localization signal. Traffic. 2010 Mar;11(3):311-23. doi: 10.1111/j. 1600-0854.2009.01028.x.
Li, C., Zhang, R., Meng, X. et al. Targeted, random mutagenesis of plant genes with dual cytosine and adenine base editors. Nat Biotechnol 38, 875-882 (2020). https://doi.org/10. 1038/s41587-019-0393-7.
Los GV, Encell LP, McDougall MG, Hartzell DD, Karassina N, Zimprich C, Wood MG, Learish R, Ohana RF, Urh M, Simpson D, Mendez J, Zimmerman K, Otto P, Vidugiris G, Zhu J, Darzins A, Klaubert DH, Bulleit RF, Wood KV. HaloTag: a novel protein labeling technology for cell imaging and protein analysis. ACS Chem Biol. 2008 Jun 20;3(6):373-82. doi: 10.1021/cb800025k.
Paul B, Chaubet L, Verver DE, Montoya G. Mechanics of CRISPR-Cas12a and engineered variants on λ-DNA. Nucleic Acids Res. 2021 Dec 24: gkab1272. doi: 10.1093/nar/gkab1272.
Peabody, DS. The RNA binding site of bacteriophage MS2 coat protein. The EMBO journal vol. 12,2 (1993): 595-600. doi: 10.1002/j. 1460-2075.1993.tb05691.x.
Rees HA, Liu DR. Base editing: precision chemistry on the genome and transcriptome of living cells. Nat Rev Genet. 2018 Dec;19(12): 770788. doi: 10.1038/S4156-018-0059-1. Erratum in: Nat Rev Genet. 2018 Oct 19.
Sambrook, J., Fritsch, E. R., & Maniatis, T. (1989). Molecular Cloning: A Laboratory Manual (2nd ed.). Cold Spring Harbor, NY: Cold Spring Harbor Laboratory Press.
Savva YA, Rieder LE, Reenan RA. The ADAR protein family. Genome Biol. 2012 Dec 28;13(12):252. doi: 10.1186/gb-2012-13-12-252.
Shan Q, Wang Y, Li J, Gao C. Genome editing in rice and wheat using the CRISPR/Cas system. Nat Protoc. 2014 Oct;9(10):2395-410. doi: 10.1038/nprot.2014.157. Epub 2014 Sep 18. PMID: 25232936.
Slaymaker IM, Gao L, Zetsche B, Scott DA, Yan WX, Zhang F. Rationally engineered Cas9 nucleases with improved specificity. Science. 2016 Jan 1;351(6268):84-8. doi: 10.1126/science.aad5227.
Yamano T, Nishimasu H, Zetsche B, Hirano H, Slaymaker IM, Li Y, Fedorova I, Nakane T, Makarova KS, Koonin EV, Ishitani R, Zhang F, Nureki O. Crystal Structure of Cpf1 in Complex with Guide RNA and Target DNA. Cell. 2016 May 5;165(4):949-62. doi: 10.1016/j.cell.2016.04.003.
Yan D, Ren B, Liu L, Yan F, Li S, Wang G, Sun W, Zhou X, Zhou H. High-efficiency and multiplex adenine base editing in plants using new TadA variants. Mol Plant. 2021 May 3;14(5):722-731. doi: 10.1016/j.molp.2021.02.007.
Yu Q, Powles SB. Resistance to AHAS inhibitor herbicides: current understanding. Pest management science vol. 70,9 (2014): 1340-50. doi: 10.1002/ps.3710.Zhang X, Chen L, Zhu B, Wang L, Chen C, Hong M, Huang Y, Li H, Han H, Cai B, Yu W, Yin S, Yang L, Yang Z, Liu M, Zhang Y, Mao Z, Wu Y, Liu M, Li D. Increasing the efficiency and targeting range of cytidine base editors through fusion of a single-stranded DNA-binding protein domain. Nat Cell Biol. 2020 Jun;22(6):740-750. doi: 10.1038/s41556-020-0518-8.

1. A method for targeted diversifying base editing of at least one target nucleic acid segment, comprising
(a) providing at least one cell or construct comprising at least one target nucleic acid segment;
(b) introducing into the target cell, or contacting with the target construct;
(i) at least one diversifying base editor (DBE), or at least one nucleic acid molecule encoding the same; and
(ii) at least one suitable guide RNA or at least one nucleic acid molecule encoding the same;
(c) allowing complex formation of (i) the at least one diversifying base editor and (ii) the at least one suitable guide RNA;
(d) obtaining at least one cell or construct comprising at least one modified target nucleic acid segment;
wherein the total base editing efficiency of introducing at least one substitution of any kind into the at least on target nucleic acid segment is at least 0.2%, 0.5%,1%, 5%, 10%, 15%, 20%, or at least 25%, wherein the upper limit is 100% or less; and/or
wherein the at least one modification of the target nucleic acid segment occurs in an extended base editing window;
and wherein the method does not comprise treatment of the human or animal body by surgery or therapy and/or a diagnostic method practised on the human or animal body, and/or processes for modifying the germ line genetic identity of human beings,
and wherein the diversifying base editor comprises a CRISPR-Cas portion originating from a Class 2 Type V CRISPR-Cas endonuclease.

2. The method of claim 1, wherein the diversifying base editor comprises a CRISPR-Cas portion originating from a Cas12a endonuclease.

3. The method of claim 1, wherein the at least one target cell is a prokaryotic cell, a bacterial cell, an archaea cell, a eukaryotic cell, an insect cell, a mammalian cell or plant cell.

4. The method of claim 1, wherein the at least one target cell is a plant cell.

5. The method of claim 1, wherein the at least one diversifying base editor comprises
(i) one or more cytosine deaminase portion(s),
(ii) one or more adenine deaminase portion(s),
(iii) one or more CRISPR-Cas portion(s),
(iv) one, two, three or more nuclear localization sequence(s); and
(v) at least one linker region.

6. The method of claim 5, wherein the at least one diversifying base editor of step (b-i) is at least one diversifying base editor in form of a fusion protein.

7. The method of claim 1, wherein the diversifying base editor comprises at least one further portion, wherein the at least one further portion is selected from an ssDNA-, ssRNA-, or dsRNA-binding protein portion, an MS2 protein portion, an affinity tag binding protein, a uracil glycosylase inhibitor portion and/or a uracil glycosylase portion, or any combination thereof.

8. The method claim 1, wherein the one or more adenine deaminase portion(s) and/or the one or more cytosine deaminase portion(s) is/are linked to at least one ssRNA-or dsRNA-binding protein portion, optionally at least one MS2 protein portion, and the at least one suitable guide RNA is adapted to allow interaction with the at least one ssRNA- or dsRNA-binding protein portion, optionally wherein the one or more adenine base editor portion and/or the one or more cytosine base editor portion is/are linked to at least one MS2 protein portion and the suitable guide RNA is adapted to comprise two MS2 stem-loops, optionally wherein the suitable guide RNA comprises a sequence selected from SEQ ID NO: 38 to SEQ ID NO: 41, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

9. The method of claim 1, wherein the diversifying base editor comprises an amino acid molecule selected from any one of SEQ ID NO: 1-27 or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the respective reference sequence.

10. An edited cell, tissue, organ, material or whole organism obtained by or obtainable by a method according to claim 1.

11. A diversifying base editor, or a diversifying base editor complex additionally comprising at least one suitable guide RNA, or at least one nucleic acid molecule encoding the same, wherein the diversifying base editor is as defined in claim 5.

12. A vector or expression construct, or more than one vectors and expression constructs, each vector and/or expression construct comprising the at least one nucleic acid molecule of claim 11, wherein different portions of the diversifying base editor are encoded on the same vector or expression construct or on different vectors or expression constructs, and/or wherein the diversifying base editor, or portions thereof, and the at least one suitable guide RNA are encoded on the same vector or expression construct or on different vectors or expression constructs.

13. A cell comprising at least one diversifying base editor or at least one diversifying base editor complex, or at least one nucleic acid molecule encoding the same, of claim 11;
or at least one vector or expression construct comprising the at least one nucleic acid molecule of claim 11 wherein the cell is a prokaryotic cell, a bacterial cell, an archaea cell, a eukaryotic cell, an insect cell, a mammalian cell, a human cell, plant cell, a plant protoplast, or a cell of, or originating from, a plant selected from wherein the at least one target cell is a plant cell of, or originating from, a plant which belongs to the superfamily Viridiplantae, monocotyledonous and dicotyledonous plants, including fodder or forage legumes, ornamental plants, food crops, trees or shrubs selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp., Averrhoa carambola, Bambusa sp., Benincasa hispida, Bertholletia excelsea, Beta vulgaris, Brassica spp., Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis, Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp., Gossypium hirsutum, Helianthus spp., Hemerocallis fulva, Hibiscus spp., Hordeum spp., Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp., Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp., Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. Tropaeolum minus, Tropaeolum majus, Vaccinium spp., Vicia spp., Vigna spp., Viola odorata, Vitis spp., Zea mays, Zizania palustris, or Ziziphus spp.

14. A kit comprising at least one diversifying base editor or at least one diversifying base editor complex, or at least one nucleic acid molecule encoding the same, of claim 11.

15. A method for for targeted directed evolution of at least one target nucleic acid segment comprising using of at least one diversifying base editor or at least one diversifying base editor complex, or at least one nucleic acid molecule encoding the same, of claim 11.

16. The method of claim 1, wherein the at least one target cell is a plant protoplast.

17. The method of claim 5, wherein the one or more CRISPR-Cas portion(s) comprise a CRISPR-Cas domain that does not cleave both strands of double-stranded DNA.

18. The method of claim 5, wherein the at least one linker region comprises one or more linker region(s) between (i) and (ii), and optionally one or more linker regions between (ii) and (iii).

19. The method of claim 6, wherein the portions (i), (ii) and (iii) are arranged, in N-terminal to C-terminal direction, in the order of (i)-(ii)-(iii) with one or more linker regions between each segment, optionally wherein one, two, three or more nuclear localization sequence(s) (iv) are located at the C-terminus of the diversifying base editor, or wherein one or more nuclear localization sequence(s) (iii) is/are located at the N-terminus and one or more nuclear localization sequence(s) (iii) is/are located at the C-terminus of the diversifying base editor.

20. The method of claim 15, wherein the method is for in planta targeted directed evolution of at least one target nucleic acid segment, for identification of at least one lead gene, for optimizing or modifying a trait in a plant, or the optimization or modification of a yield-related trait, or a disease or pathogen resistance related trait, wherein the disease is caused by, or the pathogen is selected from a virus, a bacterium, a fungus, a nematode, or an insect, or a herbicide-resistance related trait, or an abiotic-stress related trait, or a salinity or drought stress related trait.
